(19) United States
(12) Patent Application Publication
(10) Pub. No.: US 2010/0022611 A1
(43) Pub. Date: Jan. 28, 2010

(54) 1,5-Diaryl-Pyrazoles As Cannabinoid Receptor Neutral Antagonists Useful As Therapeutic Agents

(75) Inventors: Iain Robert Greig, Aberdeenshire (GB); Ruth Alexandra Ross, Aberdeenshire (GB); Roger Guy Pertwee, Aberdeenshire (GB)

Correspondence Address: SWANSON & BRATSCHUN, L.L.C., 8210 SOUTHPARK TERRACE, LITTLETON, CO, 80120, US

(73) Assignee: THE UNIVERSITY COURT OF THE UNIVERSITY OF ABERDEEN, Aberdeenshire (GB)

(21) Appl. No.: 12/525,002

(22) PCT Filed: Feb. 7, 2008
(86) PCT No.: PCT/GB08/00435

§ 371 Date: Jul. 29, 2009

(30) Foreign Application Priority Data
Feb. 14, 2007 (GB) 0702862.4

(51) Int. Cl. A61K 31/415; C07D 231/10; A61P 3/00; A61P 25/00; A61P 19/00; A61P 29/00; A61P 37/00

(52) U.S. Cl. 514/406; 548/374.1

(57) ABSTRACT

The present invention pertains to cannabinoid (CB) receptor neutral antagonists, and especially CB1 neutral antagonists, and including, for example, certain 1,5-di-aryl-pyrazole compounds. The present invention also pertains to the use of such compounds in the treatment of diseases and disorders that are ameliorated by treatment with a neutral antagonist of the cannabinoid type 1 (CB1) receptor, for example: an eating disorder; obesity; a disease or disorder characterised by an addiction component; addiction; withdrawal; smoking addiction; smoking withdrawal; drug addiction; drug withdrawal; smoking cessation therapy; a bone disease or disorder; osteoporosis, Paget's disease of bone; bone related cancer; a disease or disorder with an inflammatory or autoimmune component; rheumatoid arthritis; inflammatory bowel disease; psoriasis; a psychiatric disease or disorder; anxiety; mania; schizophrenia; a disease or disorder characterised by impairment of memory and/or loss of cognitive function; memory impairment; loss of cognitive function; Parkinson's disease; Alzheimer's disease; dementia; a cardiovascular disease or disorder; congestive heart failure; cardiac hypertrophy; and myocardial infarction.

### RELATED APPLICATION

This application is related to United Kingdom patent application number 0702862.4 filed 14 Feb. 2007, the contents of which are incorporated herein by reference in their entirety.

### TECHNICAL FIELD

The present invention pertains to cannabinoid (CB) receptor neutral antagonists, and especially CB1 neutral antagonists, and including, for example, certain 1,5-di-aryl-pyrazole compounds. The present invention also pertains to the use of such compounds in the treatment of diseases and disorders that are ameliorated by treatment with a neutral antagonist of the cannabinoid type 1 (CB1) receptor.

### BACKGROUND

Throughout this specification, including any claims which follow, unless the context requires otherwise, the word “comprise,” and variations such as “comprises” and “comprising,” will be understood to imply the inclusion of a stated integer or step or group of integers or steps, but not the exclusion of any other integer or step or group of integers or steps.

It must be noted that, as used in the specification and any appended claims, the singular forms “a,” “an,” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

This disclosure includes information that may be useful in understanding the present invention. It is not an admission that any of the information provided herein is prior art or relevant to the presently claimed invention, or that any publication specifically or implicitly referenced is prior art.

### Cannabis and Cannabinoids

The plant species Cannabis sativa L, also known as cannabis, marijuana, and Indian hemp, is of the genus Cannabis L. (hemp) and the family Cannabaceae (also Cannabidaceae) (hemp family). Two sub-species are known, ssp. indica and ssp. sativa, as well as several varieties of the latter (e.g., Purple Haze).

Cannabis is a source of fiber (hemp), oil, medicines, and narcotics (psychotropics). Most varieties contain biologically active terpenoid derivatives, such as cannabinol, isomeric tetrahydrocannabinols, and cannabidiol, collectively referred to as “cannabinoids.” A variety of derivatives and analogs of these compounds have been prepared and studied. Both the natural product Δ⁹-THC (also known as Dronabinol® and Marinol®) and the synthetic analogue Cesamet® (also known as Nabilone®) are licensed for use in the United Kingdom as antiemetics. See, for example, Goutopoulos et al., 2002.

### Cannabinoid Binding Sites

Specific cannabinoid binding sites for exogenous cannabinoids were first demonstrated in 1988. Since then, two distinct cannabinoid binding site receptors have been identified: the CB1 receptor and the CB2 receptor. CB1 is a ubiquitous receptor found in the central nervous system (CNS) and the periphery, and in both neural and non-neural tissues. The CB2 receptor has a more limited distribution, principally in cells associated with the immune system. Recently, another cannabinoid receptor has been characterised in the brain which binds anandamide and SR141716A, but not other cannabinoid receptor ligands (see, e.g., Breivogel et al., 2001). In addition, SR144528 may interact with a CB2-like receptor located on immune cells (Pertwee et al., 2002).

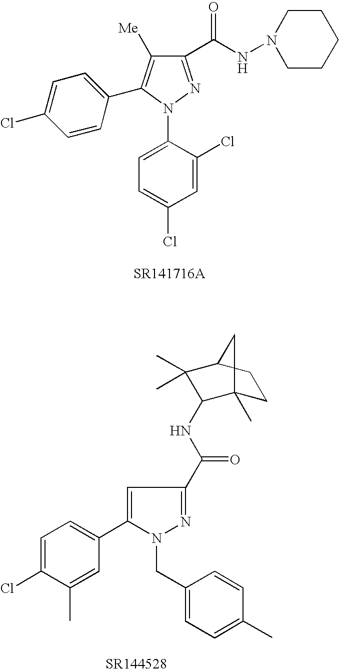

The endogenous cannabinoid (endocannabinoid) system comprises at least two receptors (CB1 and CB2), each with different localisations and functions; a family of endogenous ligands; and a specific molecular machinery for the synthesis, transport, and inactivation of these ligands. This system has been shown to have a wide range of effects in the nervous, immune, and cardiovascular systems (see, e.g., Lichtman et al., 2002; Parolaro et al., 2002; Rice et al., 2002).

### Cannabinoid Receptor Ligands

The existence of the CB1 and CB2 binding sites strongly suggested the existence of one or more endogenous ligands (endogenous cannabinoids, endocannabinoids) that exert their physiological activity upon binding to these receptors.

In 1992, the first endogenous ligand (endogenous cannabinoid, endocannabinoid), arachidonyl ethanolamide (AEA), also known as anandamide, which binds to CB1, was isolated from human brain tissue. Subsequently, a number of endogenous cannabinoids (e.g., such as those shown below) have been identified and shown to be involved in the control of various physiological functions including pain transmission, inflammation, appetite, motor function, learning and memory (see, e.g., Pertwee et al., 2002).

Cannabinoid receptor modulators are currently being investigated as a possible treatment for some of the symptoms of multiple sclerosis, neuropathic and inflammatory pain, the prevention and treatment of nausea and vomiting associated with chemotherapy, and the treatment of anorexia associated with wasting diseases.

Localisation of CB2 receptors on cells of the immune system has led to the suggestion that cannabinoid agonists may also play a role as immunosuppressive and anti-inflammatory agents. In fact, CB2 receptors have been implicated in the anti-inflammatory actions of endocananbinoids and a CB2-selective agonist has been shown to be a potent anti-inflammatory compound (see, e.g., Hanus et al., 1999).

Using DNA microarray technology it has recently been shown that activation of CB2 receptors in promyelocytic cells (HL-60) induces an up-regulation of 5 genes involved in cytokine production and regulation (IL-8, MCP-1, MIP-1β, TNFα, A20) and 4 genes involved in transcription and cell cycling (Jun B, Aldose C, BTG2, IkB-α) (see, e.g., Derocq et al., 2000). These changes are highly sensitive to cannabinoids, since significant alterations in gene expression are induced by low concentrations of agonist (10 nM). The transcriptional events reported are implicated in the cell differentiation program and suggest that CB2 receptors are important in control of the initialisation of cell maturation. Furthermore, CB2 receptor activation appears to induce conditions that promote the transition of HL-60 cells to a more monocytic/granulocytic phenotype. In addition to the observed agonist-induced enhancement of mRNA expression, a decrease in the basal levels mRNA expression was observed in the presence of the inverse agonist SR144528.

Cannabinoid receptors have been shown to play an important role in a many areas of human physiology and are treatments or potential treatments for a number of human medical conditions. Cannabinoid receptor agonists are already in use (Marinol®, Solvay; Nabilone®, Eli Lilly; Sativex®, GW Pharmaceuticals) as treatments for chemotherapy-induced nausea; for the control of pain and the treatment of spasticity in patients with multiple sclerosis; and as appetite enhancers for patients with HIV/AIDS or undergoing chemotherapy.

More recently there has been intense interest in the therapeutic properties of drugs which act as antagonists at the cannabinoid type 1 receptor (CB1). These include SR141716A (Acomplia®, Sanofi-Aventis) for which clinical trials have shown efficacy in facilitation of weight loss and cessation of smoking. The inventors have previously shown that similar compounds are able to prevent bone loss and therefore may be used in the treatment of disorders involving excessive or inappropriate bone loss, including osteoporosis, Paget's disease of bone, and bone cancers (see, e.g., Greig et al., 2004; Idris et al., 2005).

Other studies have demonstrated a role in inflammation for both the CB1 and CB2 receptors and the ability of drugs which antagonize these receptors to be used as anti-inflammatory agents in the treatment of a number of disorders, including rheumatoid arthritis, psoriasis and inflammatory bowel disease (see, e.g., Croci et al., 2003).

The traditional receptor model postulates that all receptors remain in an inactive state in the absence of agonist; this was shown to be overly simplistic in the case of cannabinoid receptors, following evidence of ligands producing effects opposite to those of an agonist (see, e.g., De Ligt et al., 2000). It is now widely accepted that cannabinoid receptors, along with other members of the GPCR superfamily including adrenoceptors, can be active even in the absence of any ligand (see, e.g., Soudijn et al., 2005). This has been termed constitutive or basal activity.

Receptor theory now proposes that at least some receptor types can exist in two interchangeable conformations, a constitutively active “on” state in which receptors are coupled to their effector mechanisms in the absence of agonist, and a constitutively inactive “off” state that is not spontaneously coupled to receptor effector mechanisms (see, e.g., Pertwee, 2005). This two-state receptor conformation model is agonist independent. However, this property is only of physiological relevance in cases where such receptors show constitutive activity.

In the case of cannabinoid receptors, which are coupled to inhibition of adenylyl cyclase, this is of great significance, because it adds two further ligand categories, inverse agonist and partial inverse agonist to the standard model. The effect of each type of ligand is described in the following Table.

TABLE 1

| Ligand class | Action | Action |
|---|---|---|
| Agonist | Stimulation of [³⁵S] GTPγS | Decrease in cAMP levels |
| | binding | |
| Partial agonist | Blocks action of agonist; | Blocks action of agonist; |
| | Stimulates sub-maximal | induces sub-maximal |
| | [³⁵S] GTPγS binding | decrease in cAMP levels |
| Antagonist | Blocks action of agonist; | Blocks action of agonist; no |
| | No change in basal | change in cAMP levels |
| | [³⁵S] GTPγS binding | |
| Partial inverse | Blocks action of agonist; | Blocks action of agonist; |
| agonist | induces sub-maximal | induces sub-maximal |
| | decrease in basal | increase in cAMP levels |
| | [³⁵S] GTPγS binding | |
| Inverse agonist | Blocks action of agonist; | Blocks action of agonist; |
| | decrease in basal | increase in cAMP levels |
| | [³⁵S] GTPγS binding | |

It has been suggested that equal binding to both receptor states is highly unlikely and that all antagonists in fact favour the “off” state of the receptor (see, e.g., De Ligt et al., 2000). As this is a response opposite to that of the agonist, these ligands have been termed inverse agonists. As with agonists, where lower efficacy ligands may activate the receptor without facilitating a maximal response, partial inverse agonists are also of importance. The ability to categorise a ligand will depend upon the degree of constitutive activity shown by the receptor and sensitivity of the assay used to measure this activity.

SR141716A has been reported to behave as both a competitive surmountable antagonist and an inverse agonist (see, e.g., Howlett et al., 2002). The lack of a sensitive assay has precluded satisfactory classification of SR141716A and other antagonists.

The inventors describe herein an improved assay which permits measurement of a significant decrease in basal [³⁵S] GTPγS binding to the CB1 receptor in response to an inverse agonist. This consequently permits determination of whether a ligand is an antagonist, a partial inverse agonist, or an inverse agonist. The inventors have used this assay to identify a class of ligands with high affinity for the CB1 receptor that show much smaller inverse agonism than SR141716A and, within the limits of the assay, are in fact true antagonists. These ligands are described herein as “neutral antagonists”.

### Advantages of Cannabinoid Receptor Neutral Antagonists

Cannabinoid receptor inverse agonists are effective in the control of obesity and encouragement of weight loss by suppression of appetite stimulating pathways. The first of these to pass through clinical trials, SR141716A (Acomplia®), allowed patients to lose 5-10% body weight each year (see, e.g., Pi-Sunyer et al., 2006). However, it also showed a high first year drop-out rate of 40-50% due to side effects such as nausea, diarrhea, dizziness, vomiting, headaches, depression, anxiety, and aggression. Weight loss tended to plateau after 34 months and patients regained the weight once treatment ceased. This finding is in agreement with animal studies in which there was a rebound effect and food intake was significantly increased in the treatment group once the drug was discontinued (see, e.g., Colombo et al., 1998; Vickers et al., 2003). These findings suggest continuous long-term therapy will be required (see, e.g., Wadman, 2006; Martindale, 2005). These are all effects that might be predicted, as they represent the opposite to the effects of cannabis itself. Prolonged use of inverse agonists has been shown to cause sensitisation of the receptor, via increased surface expression of CB1 receptors, in the same way as prolonged treatment with agonists leads to receptor internalisation and desensitization. This sensitisation may have a negative impact on drug effectiveness.

As cannabinoid inverse agonists have only recently been of interest, most of the concerns over their long-term use come from studies on other GPCRs such as the β-adrenoceptor (β-AR), histamine H2, and δ opioid receptors (see, e.g., De Ligt, 2000). Chronic administration of β-adrenoceptor (γ-AR) inverse agonists has beneficial effects in conditions in which β-blockers were traditionally contraindicated. For example, in congestive heart failure, inverse agonists of the γ-AR, produce symptomatic worsening at the onset of therapy but improve both haemodynamics and mortality with chronic use. Furthermore, in a murine model of asthma, chronic treatment with γ-AR inverse agonists increases receptor number by 7-8 fold and decreases airway resistance by 40%; these effects were not observed with neutral antagonists (see, e.g., Callaerts-Vegh et al., 2004). It is clear that chronic treatment with inverse agonists may produce upregulation of the receptor and consequent physiological changes.

Without wishing to be bound by any particular theory, the inventors believe that the use of neutral antagonists, that is, drugs which only block the effects of endogenous cannabinoids, will not cause this loss of effectiveness, and therefore have the potential to allow for continued long-term weight loss well beyond 34 months and/or without a rebound effect upon cessation of treatment. These drugs will also be of benefit in other conditions for which inverse agonists have shown potential, without the concerns over long-term usage and tolerance.

### Pyrazoles

Martin et al., 2003, describes a number of pyrazole derivatives, including O-1271, O-1272, and O-1876, shown below. However, this document provides no functional information, and instead describes all of the compounds as “CB1 antagonists” and presents circumstantial arguments in order to justify the assertion that the locomotor stimulation discussed therein is caused by antagonism rather than by inverse agonism. However, the document also uses the same locomotor stimulation data in order to show that both SR141716A and other amide derivatives act via neutral antagonism. Since SR141716A is widely accepted as an inverse agonist in most models, the locomotor stimulation model should not be accepted as a generally applicable method for differentiating between antagonism, inverse agonism, and neutral antagonism. Additionally, data presented in the document, and in an earlier publication (Wiley et al., 2001), specifically show that the ketone derivatives have lower potency (and therefore are of less interest) than their amide equivalents.

| | |
|---|---|
| O- 1271 | 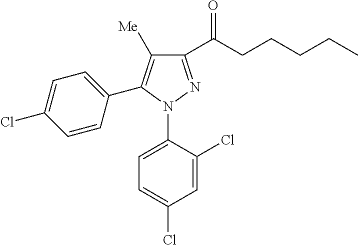 |
| O- 1272 | 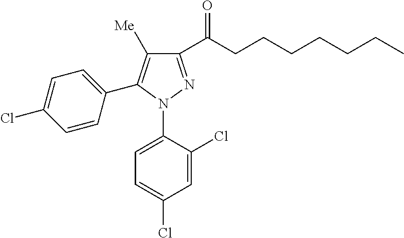 |
| O- 1876 | 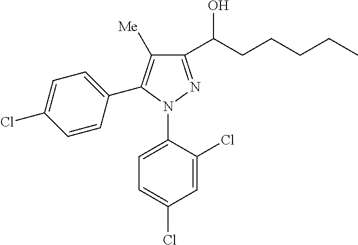 |

A number of similar pyrazole derivatives are known, but without teaching or suggestion of a biological application or any supporting biological data. Some examples are shown below.

| | |
|---|---|
| Borsche et al., 1939 | 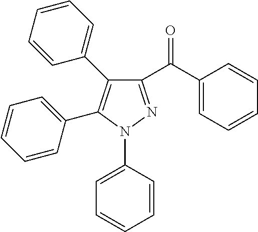 |
| Shawali, 1976 | 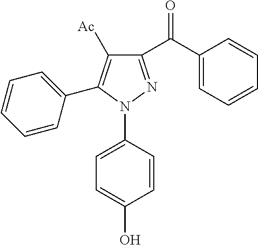 |
| Shawali, 1977 | 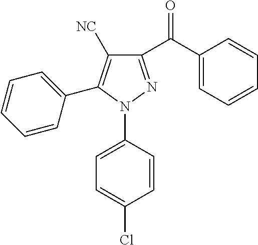 |
| Fusco et al., 1971 | 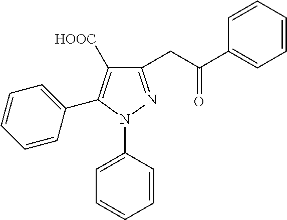 |

### SUMMARY OF THE INVENTION

One aspect of the present invention pertains to certain 1,5-di-aryl-pyrazole compounds, as described herein.

One aspect of the present invention pertains to a composition (e.g., a pharmaceutical composition) comprising a compound, as described herein, and a carrier (e.g., a pharmaceutically acceptable carrier, diluent, excipient, etc.).

One aspect of the present invention pertains to a method of making a composition (e.g., a pharmaceutical composition) comprising admixing at least one compound, as described herein, with a carrier (e.g., a pharmaceutically acceptable carrier, diluent, excipient, etc.).

One aspect of the present invention pertains to a compound as described herein for use in a method of treatment of the human or animal body by therapy.

One aspect of the present invention pertains to use of a compound as described herein in the manufacture of a medicament for use in treatment.

One aspect of the present invention pertains to a method of treatment comprising administering to a patient in need of treatment a therapeutically effective amount of a compound as described herein, preferably in the form of a pharmaceutical composition.

In one embodiment (e.g., of use in methods of therapy, of use in the manufacture of medicaments, of methods of treatment), the treatment is treatment of: a disease or disorder that is ameliorated by treatment with a neutral antagonist of the cannabinoid type 1 (CB1) receptor.

In one embodiment, the treatment is treatment of: a disease or disorder that is associated with activation of the cannabinoid type 1 (CB1) receptor.

In one embodiment, the treatment is treatment of: an eating disorder.

In one embodiment, the treatment is treatment of: obesity.

In one embodiment, the treatment is treatment of: a disease or disorder characterised by an addiction component, for example: addiction, withdrawal, smoking addiction, smoking withdrawal, drug addiction, and drug withdrawal.

In one embodiment, the treatment is smoking cessation therapy.

In one embodiment, the treatment is treatment of: a bone disease or disorder, for example: osteoporosis, Paget's disease of bone, and bone related cancer.

In one embodiment, the treatment is treatment of: a disease or disorder with an inflammatory or autoimmune component, for example: rheumatoid arthritis, inflammatory bowel disease, and psoriasis.

In one embodiment, the treatment is treatment of: a psychiatric disease or disorder, for example: anxiety, mania, and schizophrenia.

In one embodiment, the treatment is treatment of: a disease or disorder characterised by impairment of memory and/or loss of cognitive function, for example: memory impairment, loss of cognitive function, Parkinson's disease, Alzheimer's disease, and dementia.

In one embodiment, the treatment is treatment of: a cardiovascular disease or disorder, for example: congestive heart failure, cardiac hypertrophy, and myocardial infarction.

As will be appreciated by one of skill in the art, features and preferred embodiments of one aspect of the invention will also pertain to other aspects of the invention.

### BRIEF DESCRIPTION OF THE DRAWINGS

FIG. 1 is a graph showing effects of a control (DMSO), SR141716A, and several test compounds (ABD395, ABD399, ABD402 and ABD406) on electrically-evoked contractions of mouse vas deferens demonstrating that SR141716A enhances electrically-evoked contractions and is an inverse agonist, whilst the test compounds do not enhance electrically-evoked contractions and are neutral antagonists.

FIG. 2 is a graph of % stimulation versus log concentration, as obtained using a [³⁵S] GTPγS binding assay, demonstrating that SR141716A decreases basal receptor activation and is therefore an inverse agonist.

FIG. 3 is a graph of % stimulation versus log concentration, as obtained using a [³⁵S] GTPγS binding assay, demonstrating that ABD395 has no significant effect on receptor activation and is therefore a neutral antagonist.

FIG. 4 is a graph of [³⁵S] GTPγS binding to mouse brain membranes as a % of basal binding versus log concentration of CP55940, in the presence of either DMSO (vehicle) or ABD395 (300 nM), demonstrating that ABD395 is a CB1 receptor antagonist.

FIG. 5 is a graph of % stimulation versus log concentration, as obtained using a [³⁵S] GTPγS binding assay, demonstrating that ABD399 has no effect on receptor activation and is therefore a neutral antagonist.

FIG. 6 is a graph of [35S] GTPγS binding to mouse brain membranes as a % of basal binding versus log concentration of CP55940, in the presence of either DMSO (vehicle) or ABD399 (300 nM), demonstrating that ABD399 is a CB1 receptor antagonist.

FIG. 7 is a graph of % stimulation versus log concentration, as obtained using a [³⁵S] GTPγS binding assay, demonstrating that ABD402 has no effect on receptor activation and is therefore a neutral antagonist.

FIG. 8 is a graph of [³⁵S] GTPγS binding to mouse brain membranes as a % of basal binding versus log concentration of CP55940, in the presence of either DMSO (vehicle) or ABD402 (300 nM), demonstrating that ABD402 is a CB1 receptor antagonist.

FIG. 9 is a graph of % stimulation versus log concentration, as obtained using a [³⁵S]GTPγS binding assay, demonstrating that ABD406 has no effect on receptor activation and is therefore a neutral antagonist.

FIG. 10 is a graph of [³⁵S] GTPγS binding to mouse brain membranes as a % of basal binding versus log concentration of CP55940, in the presence of either DMSO (vehicle) or ABD406 (300 nM), demonstrating that ABD406 is a CB1 receptor antagonist.

### DETAILED DESCRIPTION

### Development of Neutral Antagonists

The inventors have demonstrated that replacement of the amide linkage of SR141716A and related structures with a ketone moiety reliably converts the ligand from an inverse agonist to a neutral antagonist.

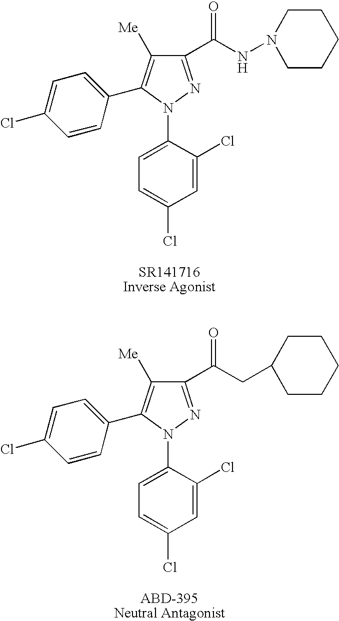

Studies have implicated a hydrogen bond formed between a lysine residue (Lys192) and the oxygen of the carboxamide in compounds such as SR141716A as being pivotal for inverse agonism to occur. Mutation at this site removes the inverse agonist properties of SR141716A, but allows it to continue to behave as an antagonist (see, e.g., Pan et al., 1998). The hydrogen bond formed is then able to stabilize a salt bridge between the lysine and an adjacent aspartate residue. This salt bridge is formed due to the presence of a pronounced kink in the receptor helix found only in the inactive state of the receptor, thereby stabilizing this inactive state and increasing its proportion relative to its active state (see, e.g., Lange et al., 2005; Hurst et al., 2002).

Without wishing to be bound by any particular theory, the inventors believe that by replacing the amide linkage with a ketone linkage, the hydrogen bonding ability of the carbonyl oxygen is retained, while its hydrogen bond acceptor properties are altered sufficiently so that it stabilizes the salt bridge to a lesser extent, and therefore no longer binds preferentially to the inactive state of the receptor.

In this way, a new class of compounds has been obtained, which act as neutral antagonists of the CB1 receptor whilst retaining high binding affinity for the receptor.

While the removal of a carboxamide group has been successfully used before as a method of removing inverse agonism (compare, for example, VCHSR and 5-(4-chlorophenyl)-1-(2,4-dichlorophenyl)-3-hexyl-1H-1,2,4-triazole), those compounds also had weaker binding to the CB1 receptor. Those compounds, and the literature in general (see, e.g., Lange et al., 2005), might possibly suggest that the carboxamide oxygen atom is involved in the property of inverse agonism and that its removal can be used to transform an inverse agonist to an antagonist. However, they do not suggest its replacement with any other group and, more importantly, do not indicate that use of a ketone group would be beneficial for retaining binding affinity.

### Compounds

One aspect of the present invention pertains to compounds of the following formula, and pharmaceutically acceptable salts, hydrates, and solvates thereof:

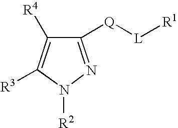

wherein:

Q is independently selected from the following groups:

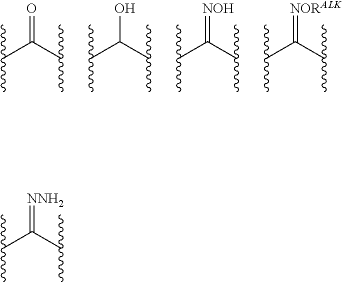

R^{ALK} is independently C₁₋₃alkyl;

L is independently a covalent bond or C₁₋₃alkylene;

R¹ is independently:

C₆₋₁₄carboaryl, and is independently unsubstituted or substituted with one or more ring substituents; or

C₅₋₁₄heteroaryl, and is independently unsubstituted or substituted with one or more ring substituents; or

C₅₋₈cycloalkyl, and is independently unsubstituted or substituted with one or more ring substituents;

R² is independently a group of the following formula, wherein each of R^{2A}, R^{2B}, R^{2C}, R^{2D}, and R^{2E} is independently —H, —Cl, —Br, or —I:

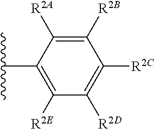

R³ is independently a group of the following formula wherein each of R^{3A}, R^{3B}, R^{3C}, R^{3D}, and R^{3E} is independently —H, —Cl, —Br, or —I:

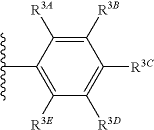

R⁴ is independently C₁₋₇alkyl.

### The Group Q

The group Q is independently selected from the following groups, wherein R^{ALK} is independently Cl₁₋₃alkyl:

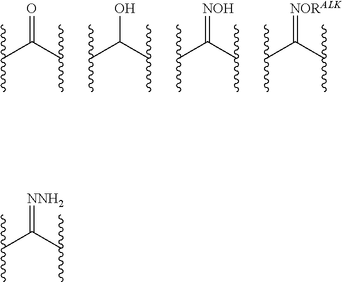

(These are, in order: a keto group; a reduced keto group; a keto group protected as an oxime; a keto group protected as alkyloxime; and a keto group protected as a hydrazide.)

In one embodiment, Q is independently selected from:

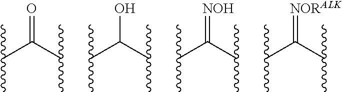

In one embodiment, Q is independently selected from:

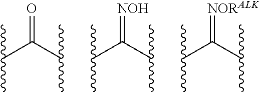

In one embodiment, Q is independently selected from:

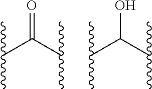

In one embodiment, Q is independently:

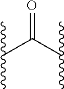

In one embodiment, R^{ALK} is independently -Me or -Et.

In one embodiment, R^{ALK} is independently -Me.

### The Group L

The group, L, is independently a covalent bond or C₁₋₃alkylene.

In one embodiment, L is independently a covalent bond.

In one embodiment, L is independently C₁₋₃alkylene.

In one embodiment, L is independently a covalent bond, —CH₂—, —CH₂CH₂—, or —CH₂CH₂CH₂—.

In one embodiment, L is independently a covalent bond, —CH₂— or —CH₂CH₂—.

In one embodiment, L is independently a covalent bond or —CH₂—.

In one embodiment, L is independently —CH₂—, —CH₂CH₂—, or —CH₂CH₂CH₂—.

In one embodiment, L is independently —CH₂— or —CH₂CH₂—.

In one embodiment, L is independently —CH₂—.

(For the avoidance of doubt, when L is a covalent bond, then the group R¹ is joined directly to the group Q. For example, when -Q- is —C(═O)— and L is a covalent bond and R¹ is phenyl, then the group -Q-L-R¹ is —C(═O)-Ph.)

### The Group R¹

The group R¹ is independently:
C₆₋₁₄carboaryl, and is independently unsubstituted or substituted with one or more ring substituents; or
C₅₋₁₄heteroaryl, and is independently unsubstituted or substituted with one or more ring substituents; or
C₅₋₈cycloalkyl, and is independently unsubstituted or substituted with one or more ring substituents.

In one embodiment, R¹ is independently:
phenyl or naphthyl, and is independently unsubstituted or substituted with one or more ring substituents; or
pyrrolyl, furanyl, thienyl, oxazolyl, isoxazolyl, thiazolyl, isothiazolyl, imidazolyl, pyrazolyl, pyridyl, or pyrimidinyl, and is independently unsubstituted or substituted with one or more ring substituents; or
benzofuranyl, isobenzofuranyl, indolyl, isoindolyl, benzimidazolyl, benzoxazolyl, benzisoxazolyl, benzodioxolyl, benzothiofuranyl, benzothiazolyl, or benzothiadiazolyl, and is independently unsubstituted or substituted with one or more ring substituents; or
quinolinyl, isoquinolinyl, benzodiazinyl, pyridopyridinyl, or quinoxalinyl, and is independently unsubstituted or substituted with one or more ring substituents; or
cyclopentyl, cyclohexyl, cycloheptyl, or cyclooctyl, and is independently unsubstituted or substituted with one or more ring substituents.

In one embodiment, R¹ is independently:
C₆₋₁₀carboaryl, and is independently unsubstituted or substituted with one or more ring substituents; or
C₅₋₁₀heteroaryl, and is independently unsubstituted or substituted with one or more ring substituents; or
C₅₋₈cycloalkyl, and is independently unsubstituted or substituted with one or more ring substituents.

In one embodiment, R¹ is independently:
C₆carboaryl, and is independently unsubstituted or substituted with one or more ring substituents; or
C₅₋₆heteroaryl, and is independently unsubstituted or substituted with one or more ring substituents; or
C₅₋₇cycloalkyl, and is independently unsubstituted or substituted with one or more ring substituents.

In one embodiment, R¹ is independently:
phenyl, and is independently unsubstituted or substituted with one or more ring substituents; or
pyrrolyl, furanyl, thienyl, oxazolyl, isoxazolyl, thiazolyl, isothiazolyl, imidazolyl, pyrazolyl, pyridyl, or pyrimidinyl, and is independently unsubstituted or substituted with one or more ring substituents; or
cyclopentyl, cyclohexyl, cycloheptyl, or cyclooctyl, and is independently unsubstituted or substituted with one or more ring substituents.

In one embodiment, R¹ is independently:
C₆₋₁₀carboaryl, and is independently unsubstituted or substituted with one or more ring substituents; or
C₅₋₈cycloalkyl, and is independently unsubstituted or substituted with one or more ring substituents.

In one embodiment, R¹ is independently:
C₆carboaryl, and is independently unsubstituted or substituted with one or more ring substituents; or
C₅₋₇cycloalkyl, and is independently unsubstituted or substituted with one or more ring substituents.

In one embodiment, R¹ is independently:
phenyl, and is independently unsubstituted or substituted with one or more ring substituents (for example, as defined below under the heading “The Group R¹-Phenyl”); or

C₅₋₇cycloalkyl, and is independently unsubstituted or substituted with one or more ring substituents (for example, as defined below under the heading “The Group R¹-Cycloalkyl”).

In one embodiment, R¹ is independently:
phenyl, and is independently unsubstituted or substituted with one or more ring substituents; or
cyclopentyl, cyclohexyl, cycloheptyl, or cyclooctyl, and is independently unsubstituted or substituted with one or more ring substituents.

In one embodiment, R¹ is independently:
phenyl, and is independently unsubstituted or substituted with one or more ring substituents.

In one embodiment, R¹ is independently:

C₅₋₈cycloalkyl, and is independently unsubstituted or substituted with one or more ring substituents.

In one embodiment, R¹ is independently:
cyclopentyl, cyclohexyl, cycloheptyl, or cyclooctyl, and is independently unsubstituted or substituted with one or more ring substituents.

### The Group R¹-Phenyl

In one embodiment, R¹ is a phenyl group, and independently is unsubstituted or substituted with one or more (e.g., 1, 2, 3, 4, 5) ring substituents.

In one embodiment, R¹ is independently a group of the following formula:

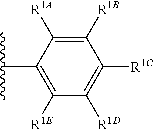

wherein each of R^{1A}, R^{1B}, R^{1C}, R^{1D}, and R^{1E} is independently —H or a ring substituent.

In one embodiment, two of R^{1A}, R^{1B}, R^{1C}, R^{1D}, and R^{1E} are each —H and the remaining three are each independently a ring substituent.

In one embodiment, three of R^{1A}, R^{1B}, R^{1C}, R^{1D}, and R^{1E} are each —H and the remaining two are each independently a ring substituent.

In one embodiment, four of R^{1A}, R^{1B}, R^{1C}, R^{1D}, and R^{1E} are —H and the remaining one is independently a ring substituent.

In one embodiment, R¹ is independently selected from:

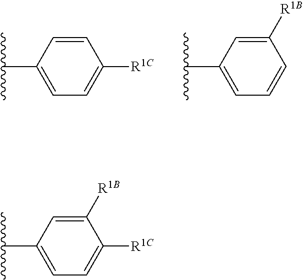

wherein each of R^{1B} and R^{1C}, if present, is independently —H or a ring substituent.

In one embodiment, R¹ is independently:

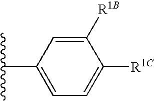

wherein each of R^{1B} and R^{1C} is independently —H or a ring substituent.

In one embodiment, each of R^{1B} and R^{1C} is independently a ring substituent.

In one embodiment, R¹ is independently:

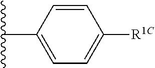

wherein R^{1C} is independently —H or a ring substituent.

In one embodiment, R^{1C} is independently a ring substituent.

In one embodiment, R¹ is independently:

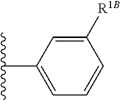

wherein R^{1B} is independently —H or a ring substituent.

In one embodiment, R^{1B} is independently a ring substituent.

In one embodiment, R¹ is independently:

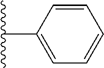

### The Group R¹-Cycloalkyl

In one embodiment, R¹ is independently:
C₅₋₈cycloalkyl, and is independently unsubstituted or substituted with one or more (e.g., 1, 2, 3, 4) ring subsituents.

In one embodiment, R¹ is independently:
C₅₋₇cycloalkyl, and is independently unsubstituted or substituted with one or more (e.g., 1, 2, 3, 4) ring subsituents.

In one embodiment, R¹ is independently:
cyclopentyl, cyclohexyl, or cycloheptyl, and is independently unsubstituted or substituted with one or more (e.g., 1, 2, 3, 4) ring subsituents.

In one embodiment, R¹ is independently a group of the following formula:

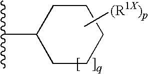

wherein:
p is independently 0, 1, 2, 3, or 4;
q is independently 0, 1, 2, or 3; and
each R^{1X}, if present, is independently a ring substituent.

In one embodiment, R¹ is independently a group of the following formula:

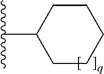

wherein q is independently 0, 1, 2, or 3.
In one embodiment, q is independently 0, 1, or 2.
In one embodiment, q is independently 0 or 1.
In one embodiment, q is independently 1 or 2.
In one embodiment, q is independently 0.
In one embodiment, q is independently 1.
In one embodiment, q is independently 2.

In one embodiment, R¹ is independently a group of the following formula:

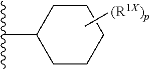

wherein:
p is independently 0, 1, 2, 3, or 4; and
each R^{1X}, if present, is independently a ring substituent.

In one embodiment, p is independently 0, 1, 2, or 3.

In one embodiment, p is independently 0, 1, or 2.

In one embodiment, p is independently 0 or 1.

In one embodiment, p is independently 1 or 2.

In one embodiment, p is independently 0.

In one embodiment, p is independently 1.

In one embodiment, p is independently 2.

In one embodiment, R¹ is independently a group of the following formula:

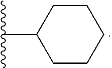

### Some Preferred Embodiments

In one embodiment, Q is —C(═O)—; L is a covalent bond; and R¹ is a phenyl group, and independently is unsubstituted or substituted with one or more (e.g., 1, 2, 3, 4, 5) ring subsituents, as in, for example:

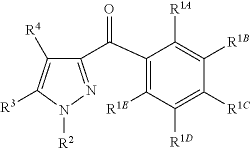

In one embodiment, Q is —C(═O)—; L is a covalent bond; and R¹ is a C₅₋₈cycloalkyl group, and independently is unsubstituted or substituted with one or more (e.g., 1, 2, 3, 4) ring subsituents, as in, for example:

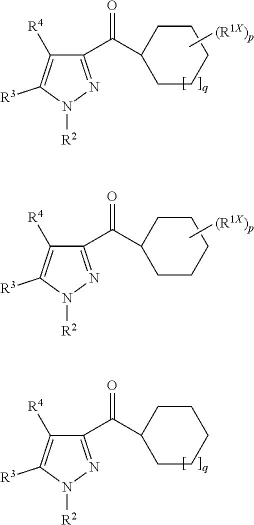

In one embodiment, Q is —C(═O)—; L is —CH₂—; and R¹ is a phenyl group, and independently is unsubstituted or substituted with one or more (e.g., 1, 2, 3, 4, 5) ring subsituents, as in, for example:

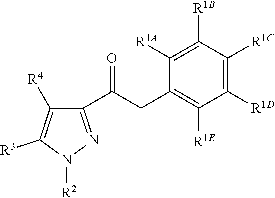

In one embodiment, Q is —C(═O)—; L is —CH₂—; and R¹ is a C₅₋₈cycloalkyl group, and independently is unsubstituted or substituted with one or more (e.g., 1, 2, 3, 4) ring subsituents, as in, for example:

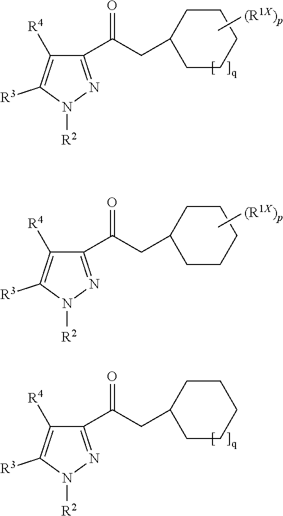

### The Group R²

The group R² is independently a group of the following formula, wherein each of R^{2A}, R^{2B}, R^{2C}, R^{2D}, and R^{2E} is independently —H, —Cl, —Br, or —I:

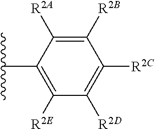

In one embodiment, two of R^{2A}, R^{2B}, R^{2C}, R^{2D}, and R^{2E} E are each —H and the remaining three are each independently —Cl, —Br, or —I.

In one embodiment, three of R^{2A}, R^{2B}, R^{2C}, R^{2D}, and R^{2E} are each —H and the remaining two are each independently —Cl, —Br, or —I.

In one embodiment, four of R^{2A}, R^{2B}, R^{2C}, R^{2D}, and R^{2E} are —H and the remaining one is independently —Cl, —Br, or —I.

In one embodiment, R² is independently selected from:

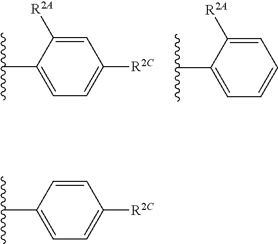

wherein each of R^{2A} and R^{2C}, if present, is independently —H, —Cl, —Br, or —I.

In one embodiment, each of R^{2A} and R^{2C}, if present, is independently —Cl, —Br, or —I.

In one embodiment, R² is independently:

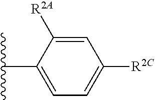

wherein each of R^{2A} and R^{2C} is independently —H, —Cl, —Br, or —I.

In one embodiment, each of R^{2A} and R^{2C} is independently —Cl, —Br, or —I.

In one embodiment, R² is independently:

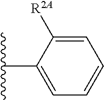

wherein R^{2A} is independently —H, —Cl, —Br, or —I.

In one embodiment, R^{2A} is independently —Cl, —Br, or —I.

In one embodiment, R² is independently:

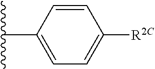

wherein R^{2C} is independently —H, —Cl, —Br, or —I.

In one embodiment, R^{2C} is independently —Cl, —Br, or —I.

In one embodiment, R² is independently:

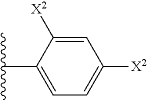

wherein each X² is independently —Cl, —Br, or —I.

In one embodiment, each X² is independently —Cl or —Br.

In one embodiment, each X² is independently —Cl.

In one embodiment, R² is independently:

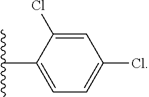

### The Group R³

The group R³ is independently a group of the following formula, wherein each of R^{3A}, R^{3A},

R^{3C}, R^{3D}, and R^{3E} is independently —H, —Cl, —Br, or —I:

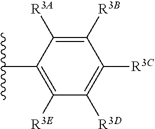

In one embodiment, two of R^{3A}, R^{3B}, R^{3C}, R^{3D}, and R^{3E} are each —H and the remaining three are each —Cl, —Br, or —I.

In one embodiment, three of R^{3A}, R^{3B}, R^{3C}, R^{3D}, and R^{3E} are each —H and the remaining two are each —Cl, —Br, or —I.

In one embodiment, four of R^{3A}, R^{3B}, R^{3C}, R^{3D}, and R^{3E} are —H and the remaining one is —Cl, —Br, or —I.

In one embodiment, R³ is independently selected from:

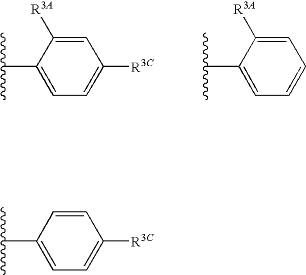

wherein each of R^{3A} and R^{3C}, if present, is independently —H, —Cl, —Br, or —I.

In one embodiment, each of R^{3A} and R^{3C}, if present, is independently —Cl, —Br, or —I.

In one embodiment, R³ is independently:

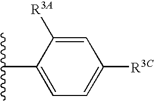

wherein each of R^{3A} and R^{3C} is independently —H, —Cl, —Br, or —I.

In one embodiment, each of R^{3A} and R^{3C} is independently —Cl, —Br, or —I.

In one embodiment, R³ is independently:

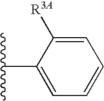

wherein R^{3A} is independently —H, —Cl, —Br, or —I.

In one embodiment, R^{3A} is independently —Cl, —Br, or —I.

In one embodiment, R³ is independently:

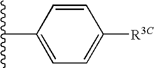

wherein R^{3C} is independently —H, —Cl, —Br, or —I.

In one embodiment, R^{3C} is independently —Cl, —Br, or —I.

> In one embodiment, R³ is independently:

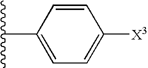

wherein X³ is independently —Cl, —Br, or —I.

In one embodiment, X³ is independently —Cl or —Br.

In one embodiment, X³ is independently —Cl.

In one embodiment, X³ is independently —Br.

In one embodiment, R³ is independently selected from:

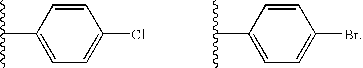

In one embodiment, R³ is independently:

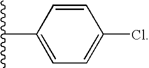

In one embodiment, R³ is independently:

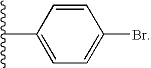

### The Group R⁴

The group R⁴ is independently C₁₋₇alkyl.

In one embodiment, R⁴ is independently C₁₋₄alkyl.

In one embodiment, R⁴ is independently -Me or -Et.

In one embodiment, R⁴ is independently -Me.

### Some Preferred Embodiments

In one embodiment, Q is —C(═O)—; L is a covalent bond; R¹ is a phenyl group, and independently is unsubstituted or substituted with one or more (e.g., 1, 2, 3, 4, 5) ring subsituents; R² is 2,4-dihalo-phenyl group; and R³ is a 4-halo-phenyl group; as in, for example:

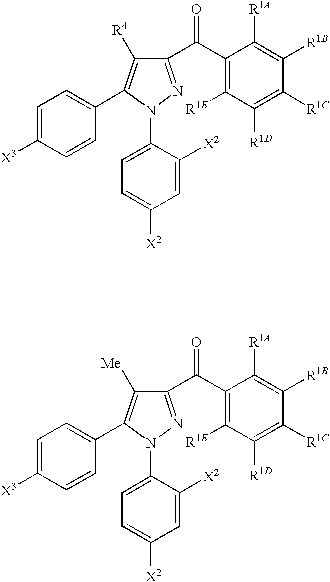

In one embodiment, Q is —C(═O)—; L is a covalent bond; R¹ is a C₅₋₈cycloalkyl group, and independently is unsubstituted or substituted with one or more (e.g., 1, 2, 3, 4) ring subsituents; R² is 2,4-dihalo-phenyl group; and R³ is a 4-halo-phenyl group; as in, for example:

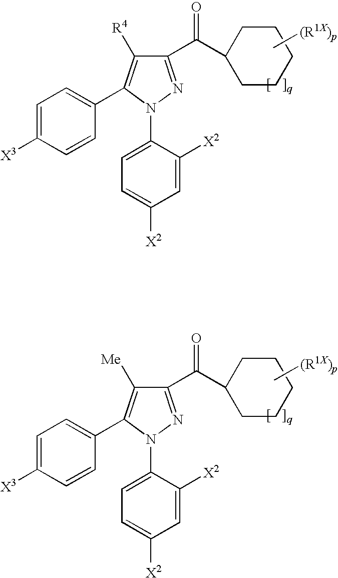

In one embodiment, Q is —C(═O)—; L is —CH₂—; R¹ is a phenyl group, and independently is unsubstituted or substituted with one or more (e.g., 1, 2, 3, 4, 5) ring subsituents; R² is 2,4-dihalo-phenyl group; and R³ is a 4-halo-phenyl group; as in, for example:

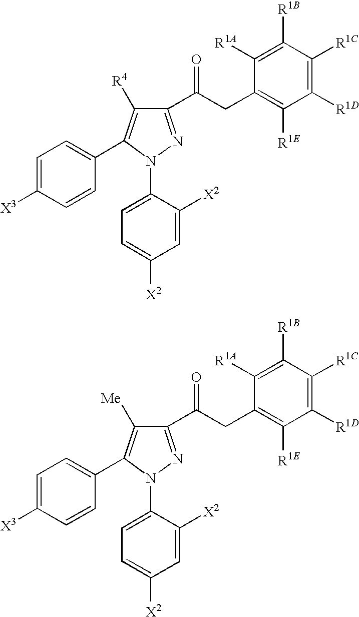

In one embodiment, Q is —C(═O)—; L is —CH₂—; R¹ is a C₅₋₈cycloalkyl group, and independently is unsubstituted or substituted with one or more (e.g., 1, 2, 3, 4) ring subsituents; R² is 2,4-dihalo-phenyl group; and R³ is a 4-halo-phenyl group; as in, for example:

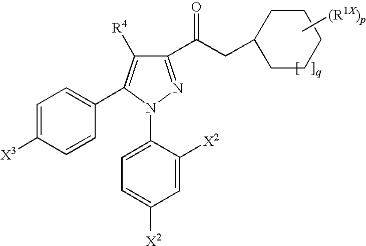

### Combinations

All compatible combinations of the embodiments described above are explicitly disclosed herein, as if each compatible combination was individually and explicitly recited.

### Ring Substituents

As discussed above, several of the groups discussed above (e.g., R¹) are independently unsubstituted or substituted with one or more (e.g., 1, 2, 3, 4, etc.) ring substituents.

The term “ring substituent”, as used herein, pertains to a substituent that is attached to a ring atom of the parent moiety.

Ring substituents, if present, may be on a ring carbon atom or a ring heteroatom. For example, when a C₅₋₆heteroaryl group includes —NH— in the aromatic ring (e.g., as in pyrrolyl, imidazolyl, pyrazolyl), this group may be N-substituted, for example N—(C₁₋₃alkyl)-substituted, for example N-(methyl)-substituted, as in, for example, N-methyl-pyrazolyl.

In one embodiment, each ring substituent is independently selected from:
(H-1) —C(═O)OH;
(H-2) —C(═O)OR^{a};
(H-3) —C(═O)NH₂, —C(═O)NHR^{a}, —C(═O)NR^{a}R^{a}, —C(═O)NR^{b}R^{c};
(H-4) —C(═O)R^{a};
(H-5) —F, —Cl, —Br, —I;
(H-6) —CN;
(H-7) —NO₂;
(H-8) —OH;
(H-9) —OR^{a};
(H-10) —SH;
(H-11) —SR^{a};
(H-12) —OC(═O)R^{a};
(H-13) —OC(═O)NH₂, —OC(═O)NHR^{a}, —OC(═O)NR^{a}R^{a}, —OC(═O)NR^{b}R^{c};
(H-14) —NH₂, —NHR^{a}, —NR^{a}R^{a}, —NR^{b}R^{c};
(H-15) —NHC(═O)R^{a}; —NR^{a}C(═O)R^{a};
(H-16) —NHC(═O)NH₂, —NHC(═O)NHR^{a}, —NHC(═O)NR^{a}R^{a}, —NHC(═O)NR^{b}R^{c}; —NR^{a}C(═O)NH₂, —NR^{a}C(═O)NHR^{a}, —NR^{s}C(═O)NR^{a}R^{a}, —NR^{a}C(═O)NR^{b}R^{c};
(H-17) —NHSO₂R^{a}, —NR^{a}SO₂R^{a};
(H-18) —SO₂R^{a};
(H-19) —OSO₂R^{a};
(H-20) —SO₂NH2, —SO₂NHR^{a}, —SO₂NR^{a}R^{a}, —SO₂NR^{b}R^{c};
(H-21) ═O;
(H-22) —CF₃; and
(H-23) —R^{d};

wherein R^{d} and each R^{a} is independently selected from:
(C-1) C₁₋₇alkyl;
(C-2) C₂₋₇alkenyl;
(C-3) C₂₋₇alkynyl;
(C-4) C₃₋₇cycloalkyl;
(C-5) C₃₋₇cycloalkenyl;
(C-6) C₃₋₁₄heterocyclyl,
(C-7) C₆₋₁₄carboaryl,
(C-8) C₅₋₁₄heteroaryl,
(C-9) C₃₋₇cycloalkyl-C₁₋₃alkylenyl,
(C-10) C₃₋₁₄heterocyclyl-C₁₋₃alkylenyl,
(C-11) C₆₋₁₄carboaryl-C₁₋₃alkylenyl, and
(C-12) C₅₋₁₄heteroaryl-C₁₋₃alkylenyl;
   wherein each C₁₋₇alkyl, C₂₋₇alkenyl, C₂₋₇alkynyl, C₃₋₇cycloalkyl, C₃₋₇cycloalkenyl, C₃₋₁₄heterocyclyl, C₆₋₁₄carboaryl, and C₅₋₁₄heteroaryl is independently unsubstituted or substituted with one or more (e.g., 1, 2, etc.) substituents selected from (H-1) through (H-22);
   and wherein R^{b} and R^{c} taken together with the nitrogen atom to which they are attached form a ring having from 3 to 7 ring atoms.

In one embodiment, each ring substituent is independently selected from:
(H′-2) —C(═O)OR^{a}′;
(H′-3) —C(═O)NH₂, —C(═O)NHR^{a}′, —C(═O)NR^{a}R^{a}′, —C(═O)NR^{b}R^{c}′;
(H′-5) —F, —Cl, —Br, —I;
(H′-6) —CN;
(H′-8) —OH;
(H″-9) —OR^{a}′;
(H′-14) —NH₂, —NHR^{a}′, —NR^{a}′R^{a}′, —NR^{b}′R^{c}′;
(H′-15) —NHC(═O)R^{a}′; —NR^{a}C(═O)R^{a}′;
(H′-17) —NHSO₂R^{a}′, —NR^{a}′SO₂R^{a}′;
(H′-18) —SO₂R^{a}′;
(H′-20) —SO₂NH₂, —SO₂NHR^{a}′, —SO₂NR^{a}′R^{a}′, —SO₂NR^{b}′R^{c}′;
(H′-22) —CF₃; and
(H′-23)-R^{d}′;
   wherein R^{d}′ and each R^{a}′ is independently selected from:
(C′-1)C₃₋₇alkyl;
(C′-4) C₃₋₇cycloalkyl;
(C′-6) C₃₋₁₄heterocyclyl,
(C′-7) C₆₋₁₄carboaryl,
(C′-8) C₅₋₁₄heteroaryl,
(C′-9) C₃₋₇cycloalkyl-C₁₋₃alkylenyl,
(C′-10) C₃₋₁₄heterocyclyl-C₁₋₃alkylenyl,
(C′-11) C₆₋₁₄carboaryl-C₁₋₃alkylenyl, and
(C′-12) C₅₋₁₄heteroaryl-C₁₋₃alkylenyl;
   wherein each C₁₋₇alkyl, C₃₋₇cycloalkyl, C₃₋₁₄heterocyclyl, C₆₋₁₄-carboaryl, and C₅₋₁₄heteroaryl is independently unsubstituted or substituted with one or more (e.g., 1, 2, etc.) substituents selected from (H′-2), (H′-3), (H′-5), (H′-6), (H′-8), (H′-9), (H′-14), (H′-15), (H′-17), (H′-18), (H′-20), and (H′-22).
   and wherein R^{b}′ and R^{c}′ taken together with the nitrogen atom to which they are attached form a ring having from 3 to 7 ring atoms.

In one embodiment, each ring substituent is independently selected from:
—C(═O)OH, —C(═O)OMe, —C(═O)OEt,
—C(═O)NH₂, —C(═O)NHMe, —C(═O)NHEt, —C(═O)NMe₂, —C(═O)NEt₂,
—SO₂Me, —SO₂OH,
—NH₂, —NHMe, —NMe₂, —NHEt, —NEt₂,
—F, —Cl, —Br, —I,
—CN,
—NO₂,
—OH, —OMe, —OEt, —O(nPr), —O(iPr), —O(cPr),
—SH, —SMe, —SEt,
-Me, -Et, -nPr, -iPr, -cPr,
—CF₃,
—OCF₃, and
═O.

In one embodiment, each ring substituent is independently selected from:
—NMe₂
—F, —Cl, —Br, —I,
—CN,
—NO₂,
—OH, —OMe, —OEt, —O(nPr), —O(iPr), —O(cPr),
—SH, —SMe, —SEt,
-Me, -Et, -nPr, -iPr, -cPr,
—CF₃, and
—OCF₃.

In one embodiment, each ring substituent is independently selected from:
—NMe₂,
—F, —Cl, —Br, —I,
—OH, —OMe, —OEt, —O(nPr), —O(iPr), —O(cPr),
-Me, -Et, -nPr, -iPr, -cPr,
—CF₃, and
—OCF₃.

In one embodiment, each ring substituent is independently selected from:
—NMe₂,
—F, —Cl, —Br, —I,
—OH, —OMe, —OEt,
-Me, -Et,
—CF₃, and
—OCF₃.

In one embodiment, each ring substituent is independently selected from:

### —F, —OMe, -Me, —CF₃, and —OCF₃.

In one embodiment, the substituents are independently selected from those substituents exemplified under the heading “Some Preferred Compounds.”

### Molecular Weight

In one embodiment, the compound has a molecular weight of 338 to 1200.

In one embodiment, the bottom of range is 340; 350; 375; 400; 425; 450.

In one embodiment, the top of range is 1100, 1000, 900; 800; 700; 600; 500.

In one embodiment, the range is 340 to 1100.

In one embodiment, the range is 340 to 1000.

In one embodiment, the range is 340 to 900.

In one embodiment, the range is 340 to 800.

In one embodiment, the range is 340 to 700.

In one embodiment, the range is 340 to 600.

In one embodiment, the range is 340 to 500.

### Combinations

All compatible combinations of the embodiments described above are explicitly disclosed herein, as if each compatible combination was individually and explicitly recited.

### Some Preferred Compounds

Some preferred compounds include the following compounds, and pharmaceutically acceptable salts, hydrates, and solvates thereof:

| | |
|---|---|
| ABD437 | 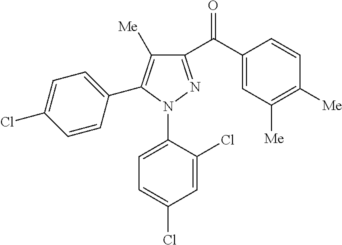 |
| ABD438 | 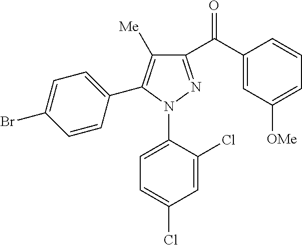 |
| ABD449 | 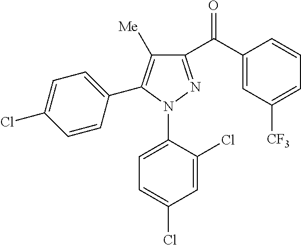 |
| ABD450 | 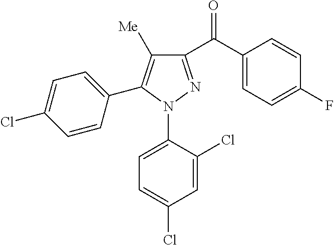 |
| ABD453 | 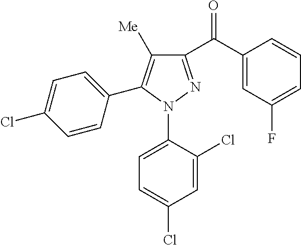 |
| ABD454 | 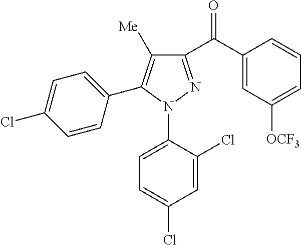 |
| ABD459 | 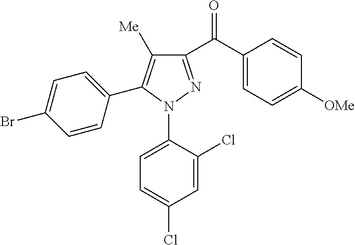 |

Some additional preferred embodiments include the following compounds, and pharmaceutically acceptable salts, hydrates, and solvates thereof:

| | |
|---|---|
| ABD406 | 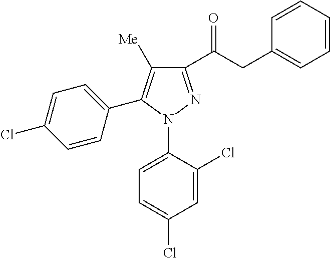 |
| ABD434 | 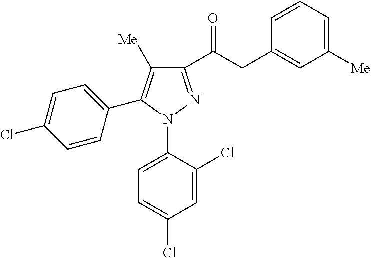 |
| ABD436 | 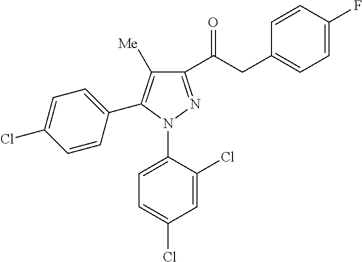 |
| ABD439 | 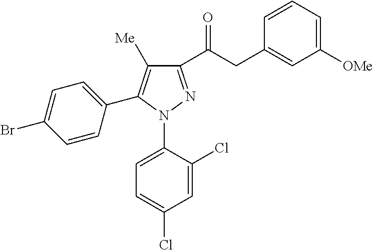 |
| ABD440 | 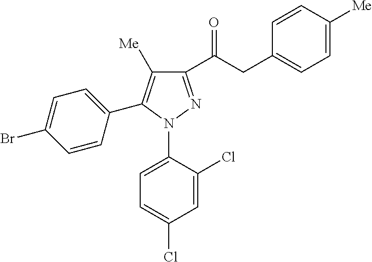 |

Some additional preferred embodiments include the following compounds, and pharmaceutically acceptable salts, hydrates, and solvates thereof:

| | |
|---|---|
| ABD395 | 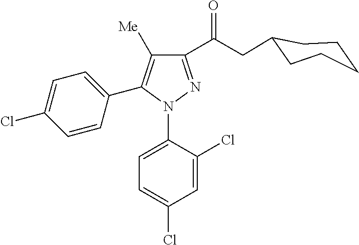 |
| ABD399 | 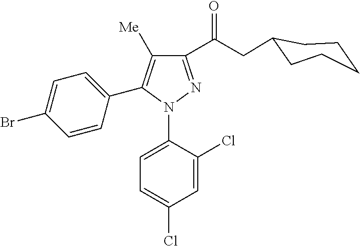 |
| ABD402 | 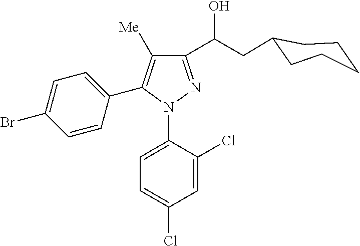 |

### Substantially Purified Forms

Another aspect of the present invention pertains to compounds, as described herein, in substantially purified form and/or in a form substantially free from contaminants.

In one embodiment, the substantially purified form is at least 50% by weight, e.g., at least 60% by weight, e.g., at least 70% by weight, e.g., at least 80% by weight, e.g., at least 90% by weight, e.g., at least 95% by weight, e.g., at least 97% by weight, e.g., at least 98% by weight, e.g., at least 99% by weight.

Unless specified, the substantially purified form refers to the compound in any stereoisomeric or enantiomeric form. For example, in one embodiment, the substantially purified form refers to a mixture of stereoisomers, i.e., purified with respect to other compounds. In one embodiment, the substantially purified form refers to one stereoisomer, e.g., optically pure stereoisomer. In one embodiment, the substantially purified form refers to a mixture of enantiomers. In one embodiment, the substantially purified form refers to an equimolar mixture of enantiomers (i.e., a racemic mixture, a racemate). In one embodiment, the substantially purified form refers to one enantiomer, e.g., optically pure enantiomer.

In one embodiment, the contaminants represent no more than 50% by weight, e.g., no more than 40% by weight, e.g., no more than 30% by weight, e.g., no more than 20% by weight, e.g., no more than 10% by weight, e.g., no more than 5% by weight, e.g., no more than 3% by weight, e.g., no more than 2% by weight, e.g., no more than 1% by weight.

Unless specified, the contaminants refer to other compounds, that is, other than stereoisomers or enantiomers. In one embodiment, the contaminants refer to other compounds and other stereoisomers. In one embodiment, the contaminants refer to other compounds and the other enantiomer.

In one embodiment, the substantially purified form is at least 60% optically pure (i.e., 60% of the compound, on a molar basis, is the desired stereoisomer or enantiomer, and 40% is the undesired stereoisomer or enantiomer), e.g., at least 70% optically pure, e.g., at least 80% optically pure, e.g., at least 90% optically pure, e.g., at least 95% optically pure, e.g., at least 97% optically pure, e.g., at least 98% optically pure, e.g., at least 99% optically pure.

### Isomers

Certain compounds may exist in one or more particular geometric, optical, enantiomeric, diastereomeric, epimeric, atropic, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and I-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers,” as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., Cl_{—}7alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enoi-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hydroxyazo, and nitro/aci-nitro.

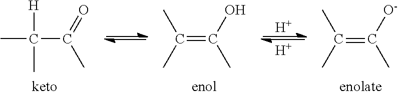

Note that specifically included in the term “isomer” are compounds with one or more isotopic substitutions. For example, H may be in any isotopic form, including 1H, 2H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof. Methods for the preparation (e.g., asymmetric synthesis) and separation (e.g., fractional crystallisation and chromatographic means) of such isomeric forms are either known in the art or are readily obtained by adapting the methods taught herein, or known methods, in a known manner.

### Salts

It may be convenient or desirable to prepare, purify, and/or handle a corresponding salt of the compound, for example, a pharmaceutically-acceptable salt. Examples of pharmaceutically acceptable salts are discussed in Berge et al., 1977, “Pharmaceutically Acceptable Salts,” J. Pharm. Sci., Vol. 66, pp. 1-19.

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, trifluoroacetic, and valeric. Examples of suitable polymeric organic anions include, but are not limited to, those derived from the following polymeric acids: tannic acid, carboxymethyl cellulose.

Unless otherwise specified, a reference to a particular compound also includes salt forms thereof.

### Solvates

It may be convenient or desirable to prepare, purify, and/or handle a corresponding solvate of the compound. The term “solvate” is used herein in the conventional sense to refer to a complex of solute (e.g., compound, salt of compound) and solvent. If the solvent is water, the solvate may be conveniently referred to as a hydrate, for example, a mono-hydrate, a di-hydrate, a tri-hydrate, etc.

Unless otherwise specified, a reference to a particular compound also includes solvate forms thereof.

### Chemical Synthesis

Several methods for the chemical synthesis of compounds of the present invention are described herein. These and/or other well known methods may be modified and/or adapted in known ways in order to facilitate the synthesis of additional compounds within the scope of the present invention.

### Compositions

One aspect of the present invention pertains to a composition comprising a compound, as described herein, and a carrier.

One aspect of the present invention pertains to a method of making a composition comprising admixing at least one compound, as described herein, with a carrier.

One aspect of the present invention pertains to a pharmaceutical composition comprising a compound, as described herein, and a pharmaceutically acceptable carrier, diluent, excipient, etc., as described below.

One aspect of the present invention pertains to a method of making a pharmaceutical composition comprising admixing at least one compound, as described herein, with a pharmaceutically acceptable carrier, diluent, excipient, etc., as described below.

### Uses

The compounds described herein are useful, for example, in the treatment of diseases and disorders that are ameliorated by treatment with a neutral antagonist of the cannabinoid type 1 (CB1) receptor, such as, for example, the diseases and disorders described below.

Similarly, the compounds described herein are useful, for example, in the treatment of diseases and disorders that are associated with activation of the cannabinoid type 1 (CB1) receptor, such as, for example, the diseases and disorders described below.

### Use in Methods of Therapy

Another aspect of the present invention pertains to a compound as described herein for use in a method of treatment of the human or animal body by therapy.

### Use in the Manufacture of Medicaments

Another aspect of the present invention pertains to use of a compound as described herein in the manufacture of a medicament for use in treatment.

In one embodiment, the medicament comprises the compound.

### Methods of Treatment

Another aspect of the present invention pertains to a method of treatment comprising administering to a patient in need of treatment a therapeutically effective amount of a compound as described herein, preferably in the form of a pharmaceutical composition.

### Diseases and Disorders Treated

In one embodiment (e.g., of use in methods of therapy, of use in the manufacture of medicaments, of methods of treatment), the treatment is treatment of: a disease or disorder that is ameliorated by treatment with a neutral antagonist of the cannabinoid type 1 (CB1) receptor.

In one embodiment (e.g., of use in methods of therapy, of use in the manufacture of medicaments, of methods of treatment), the treatment is treatment of: a disease or disorder that is associated with activation of the cannabinoid type 1 (CB1) receptor.

In one embodiment, the treatment is treatment of: an eating disorder.

In one embodiment, the treatment is treatment of: obesity.

In one embodiment, the treatment is treatment of: a disease or disorder characterised by an addiction component, for example: addiction, withdrawal, smoking addiction, smoking withdrawal, drug addiction, and drug withdrawal.

In one embodiment, the treatment is smoking cessation therapy.

In one embodiment, the treatment is treatment of: a bone disease or disorder, for example: osteoporosis, Paget's disease of bone, and bone related cancer.

In one embodiment, the treatment is treatment of: a disease or disorder with an inflammatory or autoimmune component, for example: rheumatoid arthritis, inflammatory bowel disease, and psoriasis.

In one embodiment, the treatment is treatment of: a psychiatric disease or disorder, for example: anxiety, mania, and schizophrenia.

In one embodiment, the treatment is treatment of: a disease or disorder characterised by impairment of memory and/or loss of cognitive function, for example: memory impairment, loss of cognitive function, Parkinson's disease, Alzheimer's disease, and dementia.

In one embodiment, the treatment is treatment of: a cardiovascular disease or disorder, for example: congestive heart failure, cardiac hypertrophy, and myocardial infarction.

### Treatment

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes, for example, a reduction in the rate of progress, a halt in the rate of progress, alleviation of symptoms of the condition, amelioration of the condition, and cure of the condition. Treatment as a prophylactic measure (i.e., prophylaxis) is also included. For example, use with patients who have not yet developed the condition, but who are at risk of developing the condition, is encompassed by the term “treatment.”

For example, treatment of osteoporosis includes the prophylaxis of osteoporosis, reducing the incidence of osteoporosis, alleviating the symptoms of osteoporosis, etc.

For example, treatment of an eating disorder includes, for example, management, control, and/or cessation of the eating disorder, etc.

For example, treatment of smoking addiction includes, for example, management, control, and/or cessation of smoking addiction, etc., including, e.g., smoking cessation therapy.

The term “therapeutically-effective amount,” as used herein, pertains to that amount of a compound, or a material, composition or dosage form comprising a compound, which is effective for producing some desired therapeutic effect, commensurate with a reasonable benefit/risk ratio, when administered in accordance with a desired treatment regimen.

### Combination Therapies

The term “treatment” includes combination treatments and therapies, in which two or more treatments or therapies are combined, for example, sequentially or simultaneously.

For example, the compounds described herein may also be used in combination therapies, e.g., in conjunction with other agents. Examples of treatments and therapies include, but are not limited to, chemotherapy (the administration of active agents, including, e.g., drugs, antibodies (e.g., as in immunotherapy), prodrugs (e.g., as in photodynamic therapy, GDEPT, ADEPT, etc.); surgery; radiation therapy; photodynamic therapy; gene therapy; and controlled diets.

Thus, one aspect of the present invention pertains to a compound as described herein, in combination with one or more additional therapeutic agents.

The particular combination would be at the discretion of the physician who would select dosages using his common general knowledge and dosing regimens known to a skilled practitioner.

The agents (i.e., the compound described herein, plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the therapeutic agent(s).

The agents (i.e., the compound described here, plus one or more other agents) may be formulated together in a single dosage form, or alternatively, the individual agents may be formulated separately and presented together in the form of a kit, optionally with instructions for their use.

### Other Uses

The compounds described herein may also be used as cell culture additives to provide neutral antagonism of the cannabinoid type 1 (CB1) receptor.

The compounds described herein may also be used as part of an assay (e.g., an in vitro assay), for example, in order to determine whether a candidate host is likely to benefit from treatment with the compound in question.

The compounds described herein may also be used as a standard, for example, in an assay, in order to identify other compounds, other neutral antagonists of the cannabinoid type 1 (CB1) receptor, etc.

### Kits

One aspect of the invention pertains to a kit comprising (a) a compound as described herein, or a composition comprising a compound as described herein, e.g., preferably provided in a suitable container and/or with suitable packaging; and (b) instructions for use, e.g., written instructions on how to administer the compound or composition.

The written instructions may also include a list of indications for which the active ingredient is a suitable treatment.

### Routes of Administration

The compound or pharmaceutical composition comprising the compound may be administered to a subject by any convenient route of administration, whether systemically/peripherally or topically (i.e., at the site of desired action).

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### The Subject/Patient

The subject/patient may be a chordate, a vertebrate, a mammal, a placental mammal, a marsupial (e.g., kangaroo, wombat), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human.

Furthermore, the subject/patient may be any of its forms of development, for example, a foetus.

In one preferred embodiment, the subject/patient is a human.

### Formulations

While it is possible for the compound to be administered alone, it is preferable to present it as a pharmaceutical formulation (e.g., composition, preparation, medicament) comprising at least one compound, as described herein, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents. The formulation may further comprise other active agents, for example, other therapeutic or prophylactic agents.

Thus, the present invention further provides pharmaceutical compositions, as defined above, and methods of making a pharmaceutical composition comprising admixing at least one compound, as described herein, together with one or more other pharmaceutically acceptable ingredients well known to those skilled in the art, e.g., carriers, diluents, excipients, etc. If formulated as discrete units (e.g., tablets, etc.), each unit contains a predetermined amount (dosage) of the compound.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

Suitable carriers, diluents, excipients, etc. can be found in standard pharmaceutical texts, for example, Remington's Pharmaceutical Sciences, 18th edition, Mack Publishing Company, Easton, Pa., 1990; and Handbook of Pharmaceutical Excipients. 2nd edition, 1994.

The formulations may be prepared by any methods well known in the art of pharmacy. Such methods include the step of bringing into association the compound with a carrier which constitutes one or more accessory ingredients. In general, the formulations are prepared by uniformly and intimately bringing into association the compound with carriers (e.g., liquid carriers, finely divided solid carrier, etc.), and then shaping the product, if necessary.

The formulation may be prepared to provide for rapid or slow release; immediate, delayed, timed, or sustained release; or a combination thereof.

Formulations may suitably be in the form of liquids, solutions (e.g., aqueous, non-aqueous), suspensions (e.g., aqueous, non-aqueous), emulsions (e.g., oil-in-water, water-in-oil), elixirs, syrups, electuaries, mouthwashes, drops, tablets (including, e.g., coated tablets), granules, powders, losenges, pastilles, capsules (including, e.g., hard and soft gelatin capsules), cachets, pills, ampoules, boluses, suppositories, pessaries, tinctures, gels, pastes, ointments, creams, lotions, oils, foams, sprays, mists, or aerosols.

Formulations may suitably be provided as a patch, adhesive plaster, bandage, dressing, or the like which is impregnated with one or more compounds and optionally one or more other pharmaceutically acceptable ingredients, including, for example, penetration, permeation, and absorption enhancers. Formulations may also suitably be provided in the form of a depot or reservoir.

The compound may be dissolved in, suspended in, or admixed with one or more other pharmaceutically acceptable ingredients. The compound may be presented in a liposome or other microparticulate which is designed to target the compound, for example, to blood components or one or more organs.

Formulations suitable for oral administration (e.g., by ingestion) include liquids, solutions (e.g., aqueous, non-aqueous), suspensions (e.g., aqueous, non-aqueous), emulsions (e.g., oil-in-water, water-in-oil), elixirs, syrups, electuaries, tablets, granules, powders, capsules, cachets, pills, ampoules, boluses.

Formulations suitable for buccal administration include mouthwashes, losenges, pastilles, as well as patches, adhesive plasters, depots, and reservoirs. Losenges typically comprise the compound in a flavored basis, usually sucrose and acacia or tragacanth. Pastilles typically comprise the compound in an inert matrix, such as gelatin and glycerin, or sucrose and acacia. Mouthwashes typically comprise the compound in a suitable liquid carrier.

Formulations suitable for sublingual administration include tablets, losenges, pastilles, capsules, and pills.

Formulations suitable for oral transmucosal administration include liquids, solutions (e.g., aqueous, non-aqueous), suspensions (e.g., aqueous, non-aqueous), emulsions (e.g., oil-in-water, water-in-oil), mouthwashes, losenges, pastilles, as well as patches, adhesive plasters, depots, and reservoirs.

Formulations suitable for non-oral transmucosal administration include liquids, solutions (e.g., aqueous, non-aqueous), suspensions (e.g., aqueous, non-aqueous), emulsions (e.g., oil-in-water, water-in-oil), suppositories, pessaries, gels, pastes, ointments, creams, lotions, oils, as well as patches, adhesive plasters, depots, and reservoirs.

Formulations suitable for transdermal administration include gels, pastes, ointments, creams, lotions, and oils, as well as patches, adhesive plasters, bandages, dressings, depots, and reservoirs.

Tablets may be made by conventional means, e.g., compression or moulding, optionally with one or more accessory ingredients. Compressed tablets may be prepared by compressing in a suitable machine the compound in a free-flowing form such as a powder or granules, optionally mixed with one or more binders (e.g., povidone, gelatin, acacia, sorbitol, tragacanth, hydroxypropylmethyl cellulose); fillers or diluents (e.g., lactose, microcrystalline cellulose, calcium hydrogen phosphate); lubricants (e.g., magnesium stearate, talc, silica); disintegrants (e.g., sodium starch glycolate, cross-linked povidone, cross-linked sodium carboxymethyl cellulose); surface-active or dispersing or wetting agents (e.g., sodium lauryl sulfate); preservatives (e.g., methyl p-hydroxybenzoate, propyl p-hydroxybenzoate, sorbic acid); flavours, flavour enhancing agents, and sweeteners. Moulded tablets may be made by moulding in a suitable machine a mixture of the powdered compound moistened with an inert liquid diluent. The tablets may optionally be coated or scored and may be formulated so as to provide slow or controlled release of the compound therein using, for example, hydroxypropylmethyl cellulose in varying proportions to provide the desired release profile. Tablets may optionally be provided with a coating, for example, to affect release, for example an enteric coating, to provide release in parts of the gut other than the stomach.

Ointments are typically prepared from the compound and a paraffinic or a water-miscible ointment base.

Creams are typically prepared from the compound and an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the compound through the skin or other affected areas. Examples of such dermal penetration enhancers include dimethylsulfoxide and related analogues.

Emulsions are typically prepared from the compound and an oily phase, which may optionally comprise merely an emulsifier (otherwise known as an emulgent), or it may comprises a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilisers include Tween 60, Span 80, cetostearyl alcohol, myristyl alcohol, glyceryl monostearate and sodium lauryl sulfate. The choice of suitable oils or fats for the formulation is based on achieving the desired cosmetic properties, since the solubility of the compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for intranasal administration, where the carrier is a liquid, include, for example, nasal spray, nasal drops, or by aerosol administration by nebuliser, include aqueous or oily solutions of the compound.

Formulations suitable for intranasal administration, where the carrier is a solid, include, for example, those presented as a coarse powder having a particle size, for example, in the range of about 20 to about 500 microns which is administered in the manner in which snuff is taken, i.e., by rapid inhalation through the nasal passage from a container of the powder held close up to the nose.

Formulations suitable for pulmonary administration (e.g., by inhalation or insufflation therapy) include those presented as an aerosol spray from a pressurised pack, with the use of a suitable propellant, such as dichlorodifluoromethane, trichlorofluoromethane, dichoro-tetrafluoroethane, carbon dioxide, or other suitable gases.

Formulations suitable for ocular administration include eye drops wherein the compound is dissolved or suspended in a suitable carrier, especially an aqueous solvent for the compound.

Formulations suitable for rectal administration may be presented as a suppository with a suitable base comprising, for example, natural or hardened oils, waxes, fats, semi-liquid or liquid polyols, for example, cocoa butter or a salicylate; or as a solution or suspension for treatment by enema.

Formulations suitable for vaginal administration may be presented as pessaries, tampons, creams, gels, pastes, foams or spray formulations containing in addition to the compound, such carriers as are known in the art to be appropriate.

Formulations suitable for parenteral administration (e.g., by injection), include aqueous or non-aqueous, isotonic, pyrogen-free, sterile liquids (e.g., solutions, suspensions), in which the compound is dissolved, suspended, or otherwise provided (e.g., in a liposome or other microparticulate). Such liquids may additional contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Typically, the concentration of the compound in the liquid is from about 1 ng/ml to about 10 μg/ml, for example, from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets.

### Dosage

It will be appreciated by one of skill in the art that appropriate dosages of the compounds, and compositions comprising the compounds, can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of the compound is in the range of about 100 μg to about 250 mg (more typically about 100 μg to about 25 mg) per kilogram body weight of the subject per day. Where the compound is a salt, an ester, an amide, a prodrug, or the like, the amount administered is calculated on the basis of the parent compound and so the actual weight to be used is increased proportionately.

Cannabinoid (CB) Receptor Ligands—Affinity

In general, a particular ligand which binds to a particular receptor is said to have affinity for that receptor.

A measure of affinity is often determined using a binding assay, for example, a competition or displacement assay, in which a candidate ligand competes with, or displaces, a known (or reference) ligand with a known (or reference) affinity. Such assays yield an inhibition constant (Kᵢ) for the candidate ligand. The Kᵢ value is inversely proportional to the affinity of the candidate ligand for the receptor. Thus, a low Kᵢ value signifies a high affinity. In general, a Kᵢ value of 1 μM (1000 nM) or less is considered to be a meaningful affinity for the receptor, and indicates that the candidate compounds is in fact a ligand for that receptor.

Assays for determining cannabinoid receptor affinity are well known. See, for example, Ross et al., 1999a; Ross et al., 1999b; Huffman et al., 2000; Huffman et al., 2001. For example, radio-ligand displacement assays using tissues that contain the CB1 receptor (brain, CB1 transfected cell lines) or the CB2 receptor (spleen, CB2 transfected cell lines) are common. Examples of suitable radio-labelled known (reference) ligands include tritium-labelled SR141716A (a CB1-specific receptor inverse agonist), tritium-labelled CP55940 (a CB1/CB2 receptor agonist).

### Cannabinoid Receptor Binding Assays

Cannabinoid receptor binding (and thus ligand affinity) can readily be determined by looking for displacement of a suitable known ligand by a test ligand from mouse brain and spleen membranes. Examples of suitable known ligands include tritium labelled SR141716A (a CB1-specific receptor inverse agonist) and CP55940 (a CB1/CB2 receptor agonist).

One suitable method is described next. MF1 mice are killed by cervical dislocation and the desired tissues (brain and spleen) dissected out and placed into cold centrifugation buffer (320 mM sucrose, 2 mM Tris EDTA, 5 mM MgCl₂) on ice. Tissue is then homogenized with an ultra-turrax polytron homogeniser. The homogenate is centrifuged at 1600×g for 10 minutes, the supernatant saved on ice and the pellet re-suspended in cold centrifugation buffer and centrifuged at 1600×g for 10 minutes. The supernatants are combined and centrifuged at 32000×g for 20 minutes. This supernatant is discarded and the pellet resuspended in 50 mL of Tris Buffer (50 mM Tris, 2 mM EDTA and 5 mM MgCl₂), incubated at 37° C. for 10 minutes and centrifuged at 23000×g for 20 minutes. The final pellet is resuspended in 40 mL Tris Buffer and left to stand at room temperature for 40 minutes. This solution is then centrifuged at 11000×g for 15 minutes and the pellet resuspended in assay buffer (1 mM MgCl₂, 50 mM Tris, 1 mM EDTA) to a concentration of 1 mg/mL as determined by the Lowry method (Bio-Rad Dc kit).

Radioligand binding assays are performed, for example, with the CB1 receptor inverse agonist [³H] SR141716A (0.5 nM) (brain membranes) or [³H] CP55940 (0.5 nM) (spleen membranes) in assay buffer containing 1 mg/mL BSA, the total assay volume being 500 μL. Binding is initiated by the addition of membranes (100 μg). The vehicle concentration of 0.1% DMSO is kept constant throughout. Assays are carried out at 37° C. for 60 minutes before termination by addition of ice-cold wash buffer (50 mM Tris buffer, 1 mg/mL BSA) and vacuum filtration using a 12-well sampling manifold (Brandel Cell Harvester) and Whatman GF/B glass-fibre filters that had been soaked in wash buffer at 4° C. for 24 hours. Each reaction tube is washed five times with a 4 mL aliquot of buffer. The filters are oven-dried for 60 minutes and then placed in 5 mL of scintillation fluid (Ultima Gold XR, Packard), and radioactivity quantitated by liquid scintillation spectrometry.

Specific binding is defined as the difference between the binding that occurred in the presence and absence of 1 μM unlabelled ligand and reported as a percentage of the total radio-ligand bound in brain and spleen respectively.

The concentrations of competing ligands (test compounds) to produce 50% displacement of the radioligand (IC₅₀) from specific binding sites are calculated, for example, using GraphPad Prism (GraphPad Software, San Diego). Inhibition constant (Kᵢ) values are calculated using the equation of Cheng & Prusoff (see, e.g., Cheng et al., 1973).

### Cannabinoid (CB) Receptor Ligands—Functional Characteristics (1)

Although binding studies measure the affinity of a ligand for the receptor, such studies do not indicate the functional characteristics of the ligand (that is, whether it acts as an agonist, neutral antagonist, inverse agonist, etc.).

Thus, many cannabinoid receptor ligands may also be classified according to their functional characteristics, for example, their effect upon cannabinoid receptor activity, for example, as an agonist, neutral antagonist, inverse agonist, etc.

Both CB1 and CB2 receptors belong to the G protein-coupled receptor (GPCR) super-family and are coupled to inhibition of adenylyl cyclase and activation of extracellular signal-regulated cascade (ERK). See, e.g., the review by Pertwee, 2001.

The traditional model of G protein-coupled receptor (GPCR) action is based on the premise that the binding of an agonist to the receptor is necessary for receptor activation. However, it is now clear that some receptor activation occurs spontaneously, without agonist binding, the receptors being “constitutively active.”

Cannabinoid CB1 and CB2 receptors appear to be constitutively active. A large body of evidence for this has been obtained from high expression recombinant cell lines where cannabinoid receptor inverse agonists stimulate adenylyl cyclase and inhibit ERK (see, e.g., Bouaboula et al., 1996; Bouaboula et al., 1997; Bouaboula et al., 1999). By sequestration of Gi proteins, cannabinoid inverse agonists not only inhibit constitutively active CB1/CB2 receptors but also inhibit receptor activation by other unrelated Gi-dependent receptors (see, e.g., Bouaboula et al., 1999).

In general, ligands that do not bind directly to a receptor, but do affect the receptor's function, may be described as “modulators.” There are numerous examples of so-called allosteric modulators of G-protein coupled receptors that bind to a site closely related to the receptor and modulate the function of the receptor (see, e.g., Vaulquelin et al., 2002). Such sites may exist for the cannabinoid receptors; however, none have yet been identified.

Thus, many cannabinoid receptor ligands may be further classified as:
(a) cannabinoid receptor agonists, which activate the receptor; partial agonists also activate the receptor, but with lower efficacy than a full agonist;
(b) cannabinoid receptor inverse agonists, which both block the action of the agonist and attenuate receptor-constitutive activity;
(c) cannabinoid receptor neutral antagonists, which block the action of the agonist but are ineffective on the receptor-constitutive activity; they may also be low efficacy partial agonists that behave as antagonists.

### Cannabinoid (CB) Receptor Ligands—Functional Characteristics (2)

Cannabinoid receptor ligands may be functionally characterised, for example, according to:
(1) their effect upon adenylyl cyclase activity; and/or
(2) their effect upon [³⁵S] GTPγS binding.

Thus, many cannabinoid receptor ligands may be further classified as:

(A) cannabinoid receptor agonists, which:

(i) inhibit adenylyl cyclase activity,

Inhibition of adenylyl cyclase is measured using a cyclic AMP assay (see below). Certain compounds will cause formation of cyclic AMP (i.e., stimulate cyclic AMP production) in cells and tissues. One such compound is forskolin. The stimulation of cyclic AMP production by forskolin is inhibited by cannabinoid receptor agonists. The cyclic AMP assay will yield an IC₅₀ (see methods) for cannabinoid receptor agonists. The level of inhibition of forskolin-stimulated cyclic AMP production is expressed as a percent (%) of the cyclic AMP production induced by forskolin alone. The concentration of cannabinoid receptor ligand which produces 50% inhibition (IC₅₀) of forskolin-stimulated cyclic AMP production is calculated using GraphPad Prism (GraphPad Software, San Diego). If a cannabinoid receptor ligand has an IC₅₀ value for inhibition of forskolin-stimulated cyclic AMP production of up to 10 μM (e.g., from 0.001 nM to 10 μM), then it is considered to be a cannabinoid receptor AGONIST.

and/or (ii) stimulate [³⁵S] GTPγS binding.

Agonist activation of a G-protein coupled receptor by a compound causes GTP to attach to the receptor. In this assay, the GTP is radiolabeled ([³⁵S] GTPγS) and thus the amount of GTP linked to the receptor can be measured. The amount of GTP binding to the receptor is directly proportional to the level of activation of the receptor. The [³⁵S] GTPγS binding assay measures the amount of radioactivity bound to cells and tissues. The assay will yield an EC₅₀ value for cannabinoid receptor agonists (see methods). The [³⁵S] GTPγS bound in the presence of a cannabinoid receptor agonist will increase and is expressed as a percent (%) of the specific binding. The % stimulation at each concentration of agonist is calculated and a concentration-response curve drawn using Prism (GraphPad). The concentration of agonist producing 50% stimulation of [³⁵S] GTPγS binding is defined as the EC₅₀. The Emax value is the maximum response to a given agonist. If a cannabinoid receptor ligand has an EC₅₀ value of up to 10 μM (e.g., from 0.001 nM to 10 μM) for stimulation of [³⁵S] GTPγS binding, then it is considered to be an AGONIST.

or:

(B) cannabinoid receptor inverse agonists, which:

(i) stimulate adenylyl cylase activity,

Inhibition of adenylyl cyclase is measured using a cyclic AMP assay (see below). Certain compounds will cause formation of cyclic AMP (i.e., stimulate cyclic AMP production) in cells and tissues. One such compound is forskolin. The stimulation of cyclic AMP production by forskolin is enhanced by cannabinoid receptor inverse agonists. Cannabinoid receptor inverse agonists will also stimulate the production of cyclic AMP in the absence of forskolin. A cannabinoid receptor inverse agonist will enhance forskolin-stimulated cyclic AMP production. A graph of this enhancement is drawn using GraphPad Prism (GraphPad Software, San Diego) and the EC₅₀ is the concentration of cannabinoid receptor ligand that produces a 50% stimulatory response. If a cannabinoid receptor ligand has an EC₅₀ value for stimulation of cyclic AMP production of up to 10 μM (e.g., from 0.001 nM to 10 μM), then it is considered to be a cannabinoid receptor INVERSE AGONIST.

and/or (ii) inhibit [³⁵S] GTPγS binding.

Inverse agonist activation of a G-protein coupled receptor by a compound causes GTP to detach from the receptor. In this assay, the GTP is radiolabelled ([³⁵S] GTPγS) and thus the amount of GTP linked to the receptor can be measured. The [³⁵S] GTPγS binding assay measures the amount of radioactivity bound to cells and tissues. The assay will yield an IC₅₀ value for cannabinoid receptor inverse agonists (see methods). The % inhibition is calculated for each concentration of compound and calculated and a concentration-response curve drawn using Prism (GraphPad). The concentration of inverse agonist producing 50% inhibition of [³⁵S] GTPγS binding is defined as the IC₅₀. If a cannabinoid receptor ligand has an IC₅₀ value of up to 10 μM (e.g., from 0.001 nM to 10 μM) for inhibition of [³⁵S] GTPγS binding, then it is considered to be an INVERSE AGONIST.

or:

(C) cannabinoid receptor neutral antagonists, which:

(i) block the inhibition of adenylyl cylase activity by cannabinoid receptor agonists,

As described in (A) above, the stimulation of cyclic AMP production by forskolin is inhibited by cannabinoid receptor agonist. The cyclic AMP assay will yield an IC₅₀ (see methods) for cannabinoid receptor agonists. A neutral antagonist will have no effect upon cyclic AMP production when added to cells or tissues alone. A neutral antagonist will block the inhibition of cyclic AMP production observed with an agonist (as described in (A) above). A neutral antagonist will cause the IC₅₀ for an agonist to be increased. The ratio of the IC₅₀ value in the presence and absence of an antagonist is referred to as the “dose ratio” (DR). The following formula is used to calculate the Kb value for the antagonist, where B is the concentration of antagonist: (DR−1)= (B)(Kb). The Kb value is a measure of the ability of the compound to antagonise the activation of the receptor by the agonist. A cannabinoid receptor ligand with a Kb value of up to 10 μM (e.g., from 0.001 nM to 10 μM) would be considered to be an antagonist. Note that both inverse agonists and antagonists will block the effect of agonists, but a neutral antagonist will NOT stimulate the production of cyclic AMP.

and/or (ii) block the stimulation of [³⁵S] GTPγS binding by a cannabinoid receptor agonist.

A neutral antagonist interacting with a G-protein coupled receptor will have no effect upon the GTP bound to the receptor. In this assay, the GTP is radiolabeled ([³⁵S] GTPγS) and thus the amount of GTP linked to the receptor can be measured. The [³⁵S] GTPγS binding assay measures the amount of radioactivity bound to cells and tissues. A neutral antagonist will block the stimulation of [³⁵S] GTPγS binding observed with an agonist (as described in (A) above). A neutral antagonist will cause the EC₅₀ for an agonist to be increased. The ratio of the EC₅₀ value in the absence and presence of an antagonist is referred to as the “dose ratio” (DR). The following formula is used to calculate the Kb value for the antagonist, where B is the concentration of antagonist: (DR−1)= (B)(Kb). The Kb value is a measure of the ability of the compound to antagonise the activation of the receptor by the agonist. A cannabinoid receptor ligand with a Kb value of up to 10 μM (e.g., from 0.001 nM to 10 μM) would be considered to be an antagonist. Note that both inverse agonists and antagonists will block the effect of agonists, but a neutral antagonist will NOT inhibit [³⁵S] GTPγS binding.

### Cyclic AMP Assay

Cannabinoid receptors CB1 and CB2 are coupled to inhibition of adenylyl cyclase (see, e.g., Bidault-Russell et al., 1990; Childers et al., 1996). Adenylyl cyclase is an enzyme that catalyses the production of cyclic adenosine monophosphate (AMP). Thus, activation of the receptor leads to the inhibition of the production of cyclic AMP. Certain compounds, such as forskolin, stimulate adenylyl cyclase. Accumulation of cyclic AMP is then measured using a radio-immunoassay, and is indicative of adenylyl cyclase activation. The radioimmunoassay uses radiolabeled cyclic AMP. The amount of radioactivity can be measured and will be proportional to the level of cyclic AMP that is produced. The cyclic AMP assay is performed with a phosphodiesterase inhibitor present. This is necessary because phosphodiesterase is an enzyme that rapidly breaks down cyclic AMP. An example of a phosphodiesterase inhibitor is rolipram. The cyclic AMP assay is performed using cells that contain CB1 receptors only or cells that contain CB2 receptors only (Chinese Hamster Ovary Cells or Human Embryonic Kidney Cells, respectively). The cyclic AMP assay may also be also performed with tissues that contain CB1 receptors (e.g., brain) or CB2 receptors (e.g., spleen).

The cells or tissues are incubated for 30 minutes at 37° C. with the cannabinoid receptor ligand and the phosphodiesterase inhibitor rolipram (Sigma) (50 μM) in phosphate buffered saline (PBS) containing 1 mg/ml bovine serum albumin (Sigma). The cells or tissues are then incubated for a further 30 minutes incubation with 2 μM forskolin (Sigma). The reaction is terminated by addition 0.1 M hydrochloric acid and the mixture is centrifuged in a microfuge to remove cell debris. The resulting pellet contains cell debris and the supernatant contains the [³H]cyclic AMP. A sample of a supernatant is removed and the pH is adjusted to pH 8-9 using 1 M NaOH. The cyclic AMP content is then measured using a radioimmunoassay kit ([³H] Biotrack assay TRK432, from Amersham Biosciences), following the manufacturers instructions. The amount of radioactivity in each sample is counted using a Beckman scintillation counter. The amount is cyclic AMP in each sample is calculated from the level of radioactivity.

### [³⁵S] GTPγS Assay

Activation of a G-protein coupled receptor by an agonist leads to the replacement of guanosine diphosphate (GDP) with guanosine triphosphate (GTP). The level of binding of GTP to the receptor is proportional to the level of receptor activation. The level of binding is measured by using a radiolabeled from of GTP called [³⁵S] GTPγS. Thus the radioactivity can be measured and is proportional to the amount of GTP bound to the receptor. The [³⁵S] GTPγS binding assay is performed with cells that contain CB1 receptors only or cells that contain CB2 receptors only (Chinese Hamster Ovary cells or human embryonic kidney cells, respectively). The [³⁵S] GTPγS binding assay may also be performed with tissues that contain CB1 receptors (e.g., brain) or CB2 receptors (e.g., spleen).

Cells (see above) that contain CB1 or CB2 receptors only are removed from flasks by scraping, and are re-suspended in homogenisation buffer (0.32 M sucrose/50 mM Tris), and homogenised using an Ultra-Turrex homogeniser. If tissues are used, the homogenate is prepared as for a radioligand binding assay (see above). The homogenate is diluted with Tris buffer (50 mM, pH 7.4) and centrifuged at 50,000×g for 45 minutes. Cell membranes (20 μg) (see above) are incubated in assay buffer containing 2 mg/ml fatty acid free bovine serum albumin (BSA), 20 μM GDP, and 0.1 nM [³⁵S] GTPγS (New England Nuclear). The assay buffer contains: 50 mM Tris; 10 mM MgCl₂; 100 mM NaCl; 0.2 mM EDTA at pH 7.4. Incubation times are for 90 minutes at 30° C. The reaction is terminated by the addition of 4 mL of ice-cold wash buffer (50 mM Tris, 1 mg/mL BSA, pH 7.4) followed by rapid filtration under vacuum through Whatman GF/B glass fibre filters using a 12-tube Brandel cell harvester. The filters are washed 3 times with 4 mL of wash buffer. The filters are then dried, placed in scintillation fluid, and bound radioactivity is determined by liquid scintillation counting and reported, e.g., in units of disintegrations per minute (dpm). The binding of [³⁵S] GTPγS is determined (a) in the presence of 20 μM GDP (this is the “total binding”, TB), and (b) in the presence of 10 μM [³⁵S] GTPγS (this is the “non-specific binding”, NSB). The level of binding of [³⁵S] GTPγS is reported as a percentage change with respect to basal levels. The “specific” binding (SB) of [³⁵S] GTPγS to the receptor is defined as the total binding less the non-specific binding (i.e., SB=TB−NSB), and this value is taken as 100%.

### EXAMPLES

The following examples are provided solely to illustrate the present invention and are not intended to limit the scope of the invention, as described herein.

### Chemical Synthesis

### Synthesis 1

### 5-(4-Chlorophenyl)-1-(2,4-dichlorophenyl)-4-methylpyrazole-3-carboxylic acid methoxy methyl-amide (ABD393)

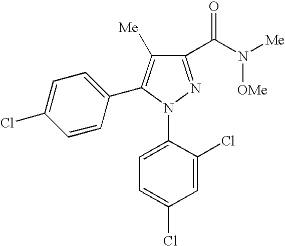

5-(4-Chlorophenyl)-1-(2,4-dichlorophenyl)-4-methylpyrazole-3-carboxylic acid chloride (1.2 g) prepared by the methods described by Barth et al. (EP 0656354 A1) and N,O-dimethyl hydroxylamine (0.4 g) were dissolved in dichloromethane (20 mL) and chilled in an ice bath. Pyridine (1 mL) was added dropwise and the mixture was stirred for 3 hours. The mixture was poured into water and the organic phase was separated, washed with water and brine, and dried. Evaporation gave an oil, which was purified by column chromatography (petrol/ethyl acetate) to give the title compound as a clear oil.

δ_{H} (CDCl₃, 250 MHz): 2.21 (3H, s), 3.44 (3H, s), 3.80 (3H, s), 7.05 (2H, d, J= 8.24 Hz), 7.21 (2H, d, J= 8.85 Hz), 7.28 (2H, d, J= 8.24 Hz) and 7.42 (1H, s).

### Synthesis 2

### 5-(4-Bromophenyl)-1-(2,4-dichlorophenyl)-4-methylpyrazole-3-carboxylic acid methoxy-methyl-amide (ABD398)

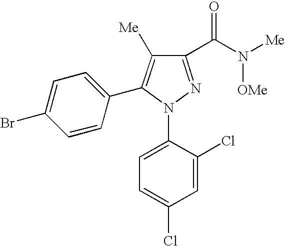

5-(4-Bromophenyl)-1-(2,4-dichlorophenyl)-4-methylpyrazole-3-carboxylic acid chloride (1.2 g) and N,O-dimethyl hydroxylamine (0.4 g) were dissolved in dichloromethane (20 mL) and chilled in an ice bath. Pyridine (1 mL) was added dropwise and the mixture was stirred for 3 hours. The mixture was poured into water and the organic phase separated, washed with water and brine, and dried. Evaporation gave an oil, which was purified by column chromatography (petrol/ethyl acetate) to give the title compound as a clear oil.

δ_{H} (CDCl₃, 250 MHz): 2.20 (3H, s), 3.43 (3H, s), 3.78 (3H, s), 6.98 (2H, d, J= 8.24 Hz), 7.17 (1H, d, J= 8.24 Hz), 7.20 (2H, d, J= 7.93 Hz) and 7.42 (2H, d, J= 8.24 Hz).

δ_{C} (CDCl₃, 62.9 MHz): 9.2, 15.3, 61.7, 65.9, 117.4, 123.1, 127.8, 127.9, 130.3, 130.6, 131.0, 131.9, 133.1, 135.8, 136.0, 141.8 and 145.5.

### Synthesis 3

### 5-(4-Chlorophenyl)-1-(2,4-dichlorophenyl)-4-methylpyrazol-3-yl]-2-cyclohexylethanone (ABD395)

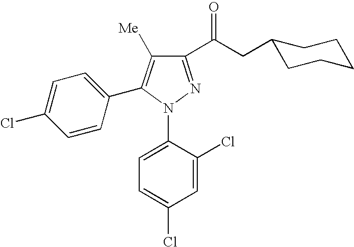

5-(4-Chlorophenyl)-1-(2,4-dichlorophenyl)-4-methylpyrazole-3-carboxylic acid methoxy-methyl-amide (1 g) was dissolved in dry THF (20 mL) and reacted with a Grignard reagent prepared from bromomethylcyclohexane (3 g) and magnesium (1 g) in dry THF (15 mL) at 0° C. The mixture was allowed to warm to room temperature and stirred for a further 2 hours. The reaction mixture was quenched with saturated NH₄Cl and the mixture was extracted with petrol. Column chromatography gave the title compound as a thick oil which gave a white solid on standing.

δ_{H} (CDCl₃, 250 MHz): 0.96 (2H, m), 1.15 (4H, m), 1.65-1.75 (7H, s), 2.31 (3H, s), 2.93 (2H, d, J= 7.02 Hz), 7.04 (2H, 6, J= 8.24 Hz), 7.24 (2H, d, J= 8.55 Hz), 7.28 (2H, d, J=8.24 Hz) and 7.40 (1H, s).

δ_{C} (CDCl₃, 62.9 MHz): 9.9, 26.3, 26.4, 33.4, 34.3, 46.9, 118.0, 127.1, 127.9, 128.9, 130.4, 130.5, 130.9, 133.1, 134.9, 136.0, 136.0, 142.8, 149.6 and 198.0.

### Synthesis 4

### 5-(4-Bromophenyl)-1-(2,4-dichlorophenyl)-4-methylpyrazol-3-yl]-2-cyclohexylethanone (ABD399)

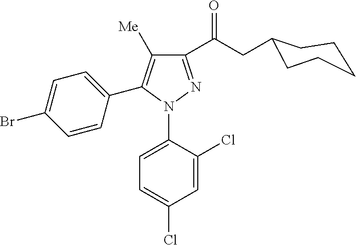

> 5-(4-Bromophenyl)-1-(2,4-dichlorophenyl)-4-methylpyrazole-3-carboxylic acid methoxy-methyl-amide (1 g) was dissolved in dry THF (20 mL) and reacted with a Grignard reagent prepared from bromomethylcyclohexane (3 g) and magnesium (1 g) in dry THF (15 mL) at 0° C. The mixture was allowed to warm to room temperature and stirred for a further 2 hours. The reaction mixture was quenched with saturated NH₄Cl and the mixture was extracted with petrol. Column chromatography gave the title compound as a thick oil which gave a white solid on standing.

δ_{H} (CDCl₃, 250 MHz): 0.94 (2H, m), 1.13 (4H, m), 1.64-1.75 (7H, s), 2.31 (3H, s), 2.93 (2H, d, J= 7.02 Hz), 6.98 (2H, d, J= 8.24 Hz), 7.24 (1H, s), 7.37 (2H, d, J= 8.24 Hz) and 7.44 (2H, d, J= 8.24 Hz).

δ_{C} (CDCl₃, 62.9 MHz): 9.9, 26.5, 33.5, 34.7, 38.0, 46.9, 118.0, 123.2, 127.6, 127.9, 130.5, 130.7, 131.1, 132.0, 133.1, 136.0, 142.8, 149.6 and 198.0

### Synthesis 5

### 1-[5-(4-Bromophenyl)-1-(2,4-dichlorophenyl)-4-methylpyrazol-3-yl]-2-cyclohexylethanol (ABD402)

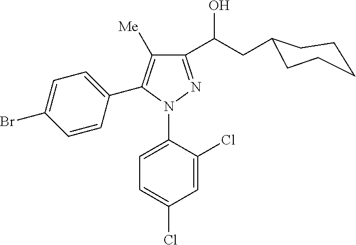

1-[5-(4-Bromophenyl)-1-(2,4-dichlorophenyl)-4-methylpyrazol-3-yl]-2-cyclohexylethanone (0.5 g) was stirred in 0.5 M NaBH₄ in diglyme (10 mL). The mixture was poured into water and extracted with ethyl acetate. Evaporation and purification by column chromatography gave the title compound as a clear oil which became a white amorphous solid on standing overnight.

δ_{H} (CDCl₃, 250 MHz): 0.95 (2H, m), 1.15 (4H, m), 1.71-1.89 (7H, s), 2.10 (3H, s), 4.95 (1H, dd, J= 8.85, 5.19 Hz), 6.98 (2H, d, J= 8.24 Hz), 7.24 (1H, s), 7.37 (2H, d, J= 8.24 Hz) and 7.42 (2H, d, J= 8.24 Hz).

δ_{C} (CDCl₃, 62.9 MHz): 8.7, 26.2, 26.4, 26.6, 32.8, 34.1, 34.2, 44.7, 66.2, 112.7, 122.7, 127.8, 130.2, 130.7, 130.9, 131.7, 133.2, 135.3, 136.4, 142.0 and 155.3.

### Synthesis 6

### 5-(4-Chlorophenyl)-1-(2,4-dichlorophenyl)-4-methylpyrazol-3-yl]-2-phenyl ethanone (ABD406)

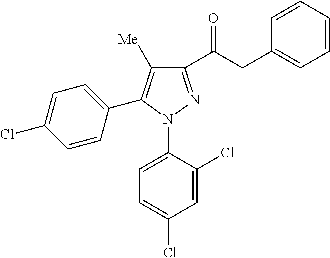

5-(4-Chlorophenyl)-1-(2,4-dichlorophenyl)-4-methylpyrazole-3-carboxylic acid methoxy-methyl-amide (1 g) was dissolved in dry THF (20 mL) and reacted with a Grignard reagent prepared from benzylbromide (3 g) and magnesium (1 g) in dry THF (15 mL) at 0° C. The mixture was allowed to warm to room temperature and stirred for a further 2 hours. The reaction mixture was quenched with saturated NH₄Cl and the mixture extracted with petrol. Column chromatography gave the title compound as a thick oil.

δ_{C} (CDCl₃, 62.9 MHz): 9.8, 46.0, 118.6, 126.7, 127.0, 128.0, 128.4, 129.0, 130.1, 130.5, 130.5, 130.9, 133.1, 134.9, 135.0, 136.0, 136.1, 143.1, 148.8 and 195.0.

### Synthesis 7

### 5-(4-Chlorophenyl)-1-(2,4-dichlorophenyl)-4-methylpyrazol-3-yl]-2-(3-methylphenyl)-ethanone (ABD434)

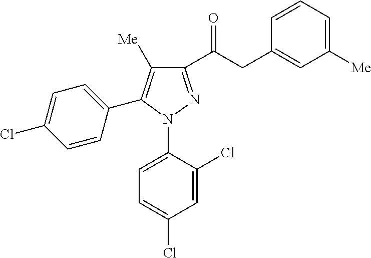

5-(4-Chlorophenyl)-1-(2,4-dichlorophenyl)-4-methylpyrazole-3-carboxylic acid methoxy-methyl-amide (0.4 g) was dissolved in dry THF (20 mL) and reacted with a Grignard reagent prepared from 3-methylbenzylbromide (3 g) and magnesium (1 g) in dry THF (15 ml) at 0° C. The mixture was allowed to warm to room temperature and stirred for a further 2 hours. The reaction was quenched with saturated NH₄Cl and the mixture extracted with petrol. Column chromatography gave the title compound as a thick oil.

δ_{H} (CDCl₃, 250 MHz): 2.29 (3H, s), 2.34 (3H, s), 4.34 (2H, s), 7.04 (4H, d, J= 8.24 Hz), 7.14 (2H, m), 7.27 (2H, m), 7.28 (2H, d, J= 8.24 Hz) and 7.46 (1H, s).

δ_{C} (CDCl₃, 62.9 MHz): 9.8, 21.4, 45.9, 118.7, 127.1, 127.5, 126.6, 128.0, 128.2, 128.3, 129.0, 129.3, 130.5, 130.9, 133.0, 134.8, 135.0, 136.0, 136.1, 138.0, 143.0, 148.8 and 195.1.

### Synthesis 8

### 5-(4-Chlorophenyl)-1-(2,4-dichlorophenyl)-4-methylpyrazol-3-yl]-2-(4-fluorophenyl)-ethanone (ABD436)

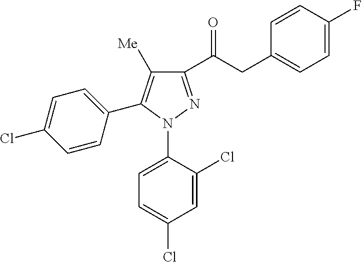

5-(4-Chlorophenyl)-1-(2,4-dichlorophenyl)-4-methylpyrazole-3-carboxylic acid methoxy-methyl-amide (0.4 g) was dissolved in dry THF (20 mL) and reacted with a Grignard reagent prepared from 4-fluorobenzylbromide (3 g) and magnesium (1 g) in dry THF (15 mL) at 0° C. The reaction mixture was allowed to warm to room temperature and stirred for a further 2 hours. The reaction was quenched with saturated NH₄Cl and the mixture extracted with petrol. Column chromatography gave the title compound as a thick oil.

δ_{H} (CDCl₃, 250 MHz): 2.28 (3H, s), 4.34 (2H, s), 6.97 (2H, d, J= 8.55 Hz), 7.02 (1H, m), 7.05 (2H, d, J= 8.24 Hz), 7.25-7.31 (5H, m) and 7.46 (1H, s).

δ_{C} (CDCl₃, 62.9 MHz): 9.8, 45.1, 115.2 (d, J= 20.5 Hz), 118.7, 126.9, 128.0, 128.9, 129.0, 130.2, 130.5, 130.9, 131.5 (d, J= 7.8 Hz), 133.0, 135.0, 136.0, 136.2, 143.2, 148.6, 161.8 (d, J=245.2 Hz) and 194.8.

### Synthesis 9

### 5-(4-Chlorophenyl)-1-(2,4-dichlorophenyl)-4-methylpyrazol-3-yl]-3,4-dimethylphenyl-methanone (ABD437)

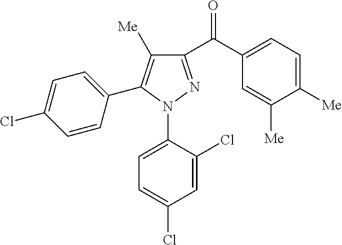

5-(4-Chlorophenyl)-1-(2,4-dichlorophenyl)-4-methylpyrazole-3-carboxylic acid methoxy-methyl-amide (1 g) was dissolved in dry THF (20 mL) and reacted with a Grignard reagent prepared from 4-bromo-o-xylene (10 g) and magnesium (3 g) in dry THF (30 mL) at 0° C. The reaction mixture was allowed to warm to room temperature and stirred for a further 2 hours. The reaction was quenched with saturated NH₄Cl and the mixture extracted with petrol. Column chromatography gave the title compound as a thick oil.

δ_{C} (CDCl₃, 62.9 MHz): 9.8, 19.5, 19.7, 119.4, 126.0, 127.2, 127.8, 128.6, 129.0, 129.6, 130.4, 130.6, 131.0, 131.9, 135.0, 135.5, 136.0, 136.5, 142.5, 142.5, 149.5 and 189.9.

### Synthesis 10

### 5-(4-Bromophenyl)-1-(2,4-dichlorophenyl)-4-methylpyrazol-3-yl]-3-methoxyphenyl-methanone (ABD438)

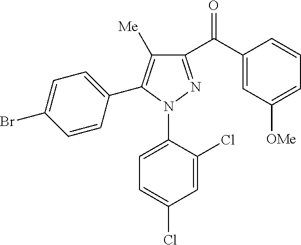

5-(4-Bromphenyl)-1-(2,4-dichlorophenyl)-4-methylpyrazole-3-carboxylic acid methoxy-methyl-amide (0.5 g) was dissolved in dry THF (20 mL) and reacted with a Grignard reagent prepared from 3-bromo-anisole (10 g) and magnesium (3 g) in dry THF (30 mL) at 0° C. The mixture was allowed to warm to room temperature and stirred for a further 10 minutes. The reaction was quenched with saturated NH₄Cl and the mixture extracted with petrol. Column chromatography gave the title compound as a thick oil.

δ_{H} (CDCl₃, 250 MHz): 2.37 (3H, s), 3.84 (3H, s), 7.05 (3H, m), 7.22 (2H, m), 7.37 (1H, t, J= 7.93 Hz), 7.46 (1H, s), 7.48 (2H, d, J= 7.32 Hz), 7.74 (1H, s) and 7.86 (1H, d, J=7.02 Hz).

δ_{C} (CDCl₃, 62.9 MHz): 10.0, 55.4, 114.7, 119.7, 123.7, 123.6, 127.5, 127.9, 129.3, 130.1, 130.4, 130.5, 131.2, 132.0, 133.1, 135.9, 136.1, 138.9 142.7, 149.2, 159.4 and 189.6.

### Synthesis 11

### 5-(4-Bromophenyl)-1-(2,4-dichlorophenyl)-4-methylpyrazol-3-yl]-2-(4-methylphenyl)-ethanone (ABD439)

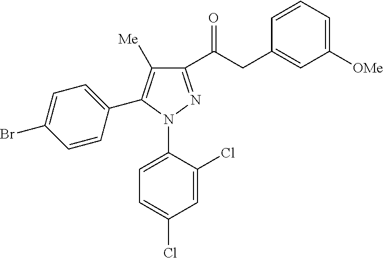

5-(4-Bromophenyl)-1-(2,4-dichlorophenyl)-4-methylpyrazole-3-carboxylic acid methoxy-methyl-amide (0.5 g) was dissolved in dry THF (20 mL) and reacted with a Grignard reagent prepared from 3-methoxybenzylbromide (3 g) and magnesium (1 g) in dry THF (15 mL) at 0° C. The reaction mixture was allowed to warm to room temperature and stirred for a further 10 minutes. The reaction was quenched with saturated NH₄Cl and the mixture extracted with petrol. Column chromatography gave the title compound as a thick oil.

δ_{H} (CDCl₃, 250 MHz): 2.29 (3H, s), 3.79 (3H, s), 4.34 (2H, s), 6.74 (1H, m), 6.78 (2H, d, J= 7.93 Hz), 6.92 (1H, m), 6.97 (2H, d, J= 8.24 Hz), 7.21 (2H, d, J= 7.63 Hz), 7.30 (2H, m) and 7.45 (1H, d, J= 7.93 Hz).

δ_{C} (CDCl₃, 62.9 MHz): 9.8, 46.0, 111.3, 112.3, 115.6, 118.7, 122.4, 123.3, 127.5, 128.0, 129.4, 130.5, 131.1, 131.9, 133.0, 135.0, 136.0, 136.1, 136.4, 143.1, 148.8, 159.6 and 194.8.

### Synthesis 12

### 5-(4-Bromophenyl)-1-(2,4-dichlorophenyl)-4-methylpyrazol-3-yl]-2-(4-methylphenyl)-ethanone (ABD440)

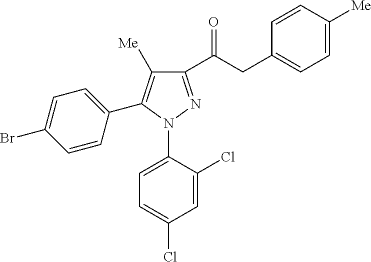

5-(4-Bromophenyl)-1-(2,4-dichlorophenyl)-4-methylpyrazole-3-carboxylic acid methoxy-methyl-amide (1 g) was dissolved in dry THF (20 mL) and reacted with a Grignard reagent prepared from 4-methylbenzylbromide (10 g) and magnesium (1 g) in dry THF (15 mL) at 0° C. The reaction mixture was allowed to warm to room temperature and stirred for a further 10 minutes. The reaction was quenched with saturated NH₄Cl and the mixture extracted with petrol. Column chromatography gave the title compound as a thick oil.

δ_{H} (CDCl₃, 250 MHz): 2.27 (3H, s), 2.31 (3H, s), 4.32 (2H, s), 6.97 (2H, d, J= 8.55 Hz), 7.12 (2H, d, J= 7.93 Hz), 7.23-7.30 (4H, m), 7.44 (2H, d, J= 8.55 Hz) and 7.46 (1H, s).

δ_{C} (CDCl₃, 62.9 MHz): 9.8, 21.1, 45.6, 118.6, 123.2, 127.6, 127.9, 129.2, 129.9, 130.5, 130.7, 131.1, 131.7, 131.9, 132.0, 133.0, 133.1, 136.0, 136.1, 142.8, 148.8 and 194.8.

### Synthesis 13

### 5-(4-Chlorophenyl)-1-(2,4-dichlorophenyl)-4-methylpyrazol-3-yl]-3-trifluoromethylphenyl-methanone (ABD449)

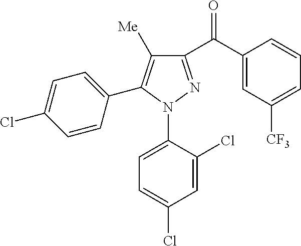

5-(4-Chlorophenyl)-1-(2,4-dichlorophenyl)-4-methylpyrazole-3-carboxylic acid methoxy-methyl-amide (0.5 g) was dissolved in dry THF (20 mL) and reacted with a Grignard reagent prepared from 3-trifluoromethyl-iodobenzene (5 g) and magnesium (3 g) in dry THF (30 mL) at 0° C. The reaction mixture was allowed to warm to room temperature and stirred for a further 2 hours. The reaction was quenched with saturated NH₄Cl and the mixture extracted with petrol. Column chromatography gave the title compound as a thick oil

δ_{H} (CDCl₃, 250 MHz): 2.40 (3H, s), 7.11 (2H, d, J= 8.55 Hz), 7.20 (1H, s), 7.24 (1H, t, J=8.55 Hz), 7.33 (2H, d, J= 8.55 Hz), 7.48 (1H, d, J= 1.81 Hz), 7.60 (1H, t, J= 7.63 Hz), 7.80 (1H, d, J= 7.63 Hz), 8.43 (1H, d, J= 7.63 Hz) and 8.53 (1H, s)

δ_{C} (CDCl₃, 62.9 MHz): 9.9, 120.0, 126.9, 127.9, 128.8, 129.1, 130.3, 130.5, 130.9, 133.0, 133.7, 135.2, 135.8, 136.1, 138.3, 142.8, 148.6 and 188.1

### Synthesis 14

### 5-(4-Chlorophenyl)-1-(2,4-dichlorophenyl)-4-methylpyrazol-3-yl]-4-fluorophenyl-methanone (ABD450)

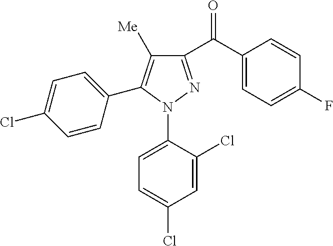

5-(4-Chlorophenyl)-1-(2,4-dichlorophenyl)-4-methylpyrazole-3-carboxylic acid methoxy-methyl-amide (0.5 g) was dissolved in dry THF (20 mL) and reacted with a Grignard reagent prepared from 4-fluoro-bromobenzene (5 g) and magnesium (3 g) in dry THF (30 mL) at 0° C. The reaction mixture was allowed to warm to room temperature and stirred for a further 2 hours. The reaction was quenched with saturated NH₄Cl and the mixture extracted with petrol. Column chromatography gave the title compound as a thick oil which became an amorphous solid on standing.

δ_{C} (CDCl₃, 62.9 MHz): 10.0, 115.3 (d, J= 21.5 Hz), 119.8, 127.0, 127.9, 129.0, 130.5 (d, J= 5.9 Hz), 131.0, 133.4, 133.5, 134.0, 134.0, 135.1, 135.9, 136.1, 142.7, 149.0, 165.7 (d, J=254.0 Hz) and 187.8.

### Synthesis 15

### 5-(4-Chlorophenyl)-1-(2,4-dichlorophenyl)-4-methylpyrazol-3-yl]-3-fluorophenyl-methanone (ABD453)

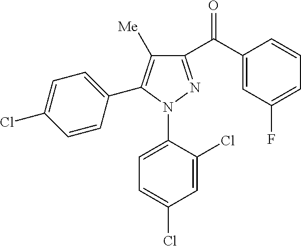

5-(4-Chlorophenyl)-1-(2,4-dichlorophenyl)-4-methylpyrazole-3-carboxylic acid methoxy-methyl-amide (0.5 g) was dissolved in dry THF (20 mL) and reacted with a Grignard reagent prepared from 3-fluoro-bromobenzene (5 g) and magnesium (3 g) in dry THF (30 mL) at 0° C. The reaction mixture was allowed to warm to room temperature and stirred for a further 2 hours. The reaction was quenched with saturated NH₄Cl and the mixture extracted with petrol. Column chromatography gave the title compound as a thick oil which became an amorphous solid on standing.

δ_{C} (CDCl₃, 62.9 MHz): 10.0, 117.6 (d, J=25.4 Hz), 119.7 (d, J= 22.5 Hz), 119.9, 126.4, 126.9, 127.9, 129.0, 129.8 (d, J=7.8 Hz), 130.5, 130.5, 130.9, 133.1, 135.2, 135.9, 136.1, 139.6 (d, J= 6.9 Hz), 142.8, 148.8, 162.4 (d, J= 247.0 Hz) and 188.9

### Synthesis 16

### 5-(4-Chlorophenyl)-1-(2,4-dichlorophenyl)-4-methylpyrazol-3-yl]-3-trifluoromethoxyphenyl-methanone (ABD454)

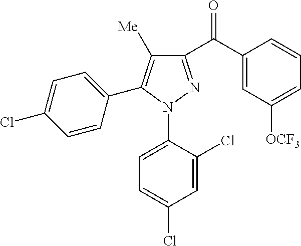

5-(4-Chlorophenyl)-1-(2,4-dichlorophenyl)-4-methylpyrazole-3-carboxylic acid methoxy-methyl-amide (0.5 g) was dissolved in dry THF (20 mL) and reacted with a Grignard reagent prepared from 3-trifluoromethoxy-bromobenzene (5 g) and magnesium (3 g) in dry THF (30 mL) at 0° C. The reaction mixture was allowed to warm to room temperature and stirred for a further 2 hours. The reaction was quenched with saturated NH₄Cl and the mixture extracted with petrol. Column chromatography gave the title compound as a thick oil which became an amorphous solid on standing.

δ_{C} (CDCl₃, 62.9 MHz): 9.9, 108.5, 112.5, 114.0, 120.1, 123.2, 125.3, 126.7, 128.0, 129.1, 130.4, 130.5, 130.9, 133.0, 135.4, 135.6, 136.4, 139.3, 143.2, 148.5, 157.1 and 188.7.

### Synthesis 17

### 5-(4-Bromophenyl)-1-(2,4-dichlorophenyl)-4-methylpyrazol-3-yl]-4-methoxyphenyl-methanone (ABD459)

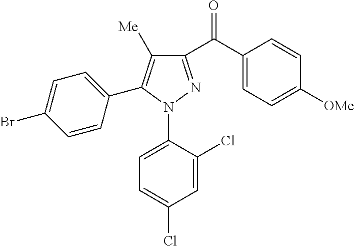

5-(4-Bromophenyl)-1-(2,4-dichlorophenyl)-4-methylpyrazole-3-carboxylic acid methoxy-methyl-amide (1 g) was dissolved in dry THF (20 mL) and reacted with a Grignard reagent prepared from 4-methoxy-bromobenzene (5 g) and magnesium (3 g) in dry THF (30 mL) at 0° C. The reaction mixture was allowed to warm to room temperature and stirred for a further 2 hours. The reaction was quenched with saturated NH₄Cl and the mixture extracted with petrol. Column chromatography gave the title compound as white powder.

δ_{H} (CDCl₃, 250 MHz): 2.36 (3H, s), 3.86 (3H, s), 6.95 (2H, d, J= 8.8 Hz), 7.04 (2H, d, J=8.2 Hz), 7.24 (1H, s), 7.48 (2H, d, J= 8.2 Hz), 7.48 (2H, d, J= 8.2 Hz) and 8.26 (2H, d, J= 8.8 Hz).

δ_{C} (CDCl₃, 62.9 MHz): 9.9, 55.5, 113.5, 119.4, 123.3, 127.7, 127.9, 130.4, 130.6, 131.2, 131.9, 133.1, 135.9, 136.0, 142.5, 149.5, 163.5 and 188.1.

### Biological Methods

Binding Assay—Mouse vas deferens

### Methods

The vas deferens (or ductus deferens) is a muscular tube, approximately 3 mm in diameter and 30 cm in length, connecting the left and right epididymis to the ejaculatory ducts in order to move sperm. It is bound by connective tissue with an ample supply of blood vessels, nerves, and lymphatics. This in vitro bioassay exploits the expression of CB1 receptors on the presynaptic nerve terminals of this tubular structure.

The animals used in the study were male albino MF1 mice bred and were housed six to eight per-cage and were kept in a temperature-controlled room which was maintained on a fixed light-dark cycle. All mice were given free access to food and water. Subjects used in the study were a minimum of four weeks of age. Each mouse was stunned by striking the back of the head and killed by dislocation of the neck (cervical vertebrae). Following the killing, the mouse body weight was determined. A transverse incision, approximately of 1.5 cm, was made in the skin with the aid of dissection scissors. A similar-sized transverse incision was made through the lower abdominal wall. Careful removal of the adipose tissue on the left side revealed the left testis. This was used to identify the vas deferens, which is attached to the testis via the epididymis. Gripping the epididymis with forceps, the vas deferens was cut free first from the testis and then from the connective tissue. The isolated vas deferens was then removed from the mouse by cutting through its prostatic end. This procedure was repeated on the right testis. It was important that throughout the latter two stages of this dissection, care was taken to ensure the vas deferens was not overstretched. In addition to their vas deferens, these animals were euthanized for use of their brain and small intestine by fellow researchers in different research fields. This minimisation of the number of animals euthanized complied with the European Community guidelines.

During transit, the two isolated vas deferens from each mice were kept moist in a glass vial filled with warm modified Mg²⁺-free Krebs' solution. Before setting the tissue up in an organ bath, further removal of connective tissue, mesentery, and the epididymis was performed; cotton thread was tied securely to both end of the vas deferens.

The one-tailed thread was attached to the Pioden UF1 isometric transducer (Harvard Apparatus) and the two-tailed thread hung out of the bottom of the bath. The latter served as an anchor point for applying tension. The tissue was mounted vertically in the 4 mL organ bath ensuring that neither the tissue nor the thread was touching any part of the organ bath and that it was in line with the Pioden UF1 isometric transducer. The isolated tissue was placed under a resting tension of 0.5 g. Contractile activity was recorded using the isometric transducer and the output monitored by a computer connected to the data recording and analysis system (MacLab or PowerLab). The tissues were then ready for stimulation.

The following Table shows the electrical stimulation conditions on the Grass S48 and Grass S88 stimulators (Grass Medical Instruments, Quincy, Mass.) necessary to generate the electrical stimuli. The stimuli were applied through a positive stainless steel electrode (anode) attached to the lower end of each bath and a negative platinum electrode (cathode) attached to the upper end. The electrical stimuli was amplified by a Med-Lab channel attenuator (Stag instruments, Chalgrove, Oxford, UK), and then divided to yield separate outputs to the eight organ baths via a Med-Lab StimuSplitter.

TABLE 1
Electrical Stimulation Conditions on Grass S48 Stimulator

| Parameter | Setting | Units |
|---|---|---|
| Train frequency | 0.1 | Hz |
| Pulse duration | 0.5 | ms |
| Pulse frequency | 5 | Hz |
| Delay | 0.01 | ms |
| Duration | 0.5 | ms |
| Voltage | 100% supramaximal | V |

After placement in an organ bath, each tissue was electrically stimulated over a period of 10 minutes, starting with a submaximal voltage and systematically increasing this output until a supramaximal voltage was achieved (110%). Electrical stimulation was then stopped and the tissue allowed to rest for 10 minutes before subjecting it to further electrical stimulation for 2 minutes. This cycle of 10 minutes of rest followed by 2 minutes of stimulation was repeated until consistent twitch amplitudes were obtained.

The equilibration procedure was followed by a 10 minute stimulation-free period. Tissues were then stimulated for 10 minutes after which the stimulator was switched off, and the test compound or its vehicle, DMSO, was added. The tissues were stimulated for the final 2 minutes of the 30 minute exposure to the test compound or DMSO. In experiments with agonist, the stimulator was once again switched off and the first addition of agonist made. Additions of all agonists used were made cumulatively at 15 minute intervals without washout, the tissues being stimulated for the final 2 minute of exposure of each concentration of the agonist (i.e., 15 minute dose cycle). Test compounds were added in a volume of 10 μL

### Analysis of Data

Values were expressed as the mean, and variability is expressed as S.E.M or as 95% confidence limits (CL). Analysis of the recorded data involved measuring the height of the last six contractions produced during each 2 minutes stimulation period. Inhibition of electrically evoked contractions was expressed as a percentage by comparing the amplitudes of the contractions after each addition of agonist with those immediately before the first addition of agonist. For all experiments with agonist, concentration-response curves were generated and the data fitted by non-linear regression analysis using GraphPad Prism (GraphPad Software, San Diego, Calif., USA). Data were fitted into the following four-parameter logistic equation:

Y=basal+(Eₘₐₓ−basal)/(1+[10^{log EC50-x]}^{nH}) (1)

wherein Y denotes effect; Eₘₐₓ and basal denote the upper and lower asymptotes, respectively; log EC₅₀ denotes the negative logarithm of the effective concentration of agonist required to elicit a 50% response; and nH denotes the Hill slope.

The precision of the EC₅₀ value obtained was governed by how well the data defined both the minimum and maximum responses. The latter was ensured by GraphPad Prism having the ability to configure the fit. Concentration-ratio values and their 95% confidence limits were determined by symmetrical (2+2) dose parallel line assays, by use of responses to pairs of agonist concentrations located on the steepest part of each log concentration-response curve (see, for example, Pertwee et al., 1996). The concentration-ratio is defined as the ratio by which the agonist concentration must be increased in the presence of antagonist in order to restore a given level of response, usually standardised at 50%. This parameter can be expressed by the following equation:

EC₅₀′/EC₅₀ (2)

wherein EC₅₀′ denotes the concentration of agonist producing half the maximal response in the presence of antagonist; and EC₅₀ denotes the concentration of agonist producing half the maximal response in the absence of antagonist.

The symmetrical (2+2) dose parallel line assay also evaluated whether or not the dextral shift deviated significantly from parallelism. A p value>0.2 assumed that the two lines were parallel. A requirement of the symmetrical (2+2) dose parallel line assay is that the value of n (sample size) is identical for the two concentration response curves being analysed.

The dissociation constant (K_{D}) values of SR141716A and its analogues, determined from experiments with CP 55940, were each calculated by substituting a single concentration-ratio value into the following Schild equation:

x−1=[B]/K_{D} (3)

wherein x denotes the concentration-ratio; and [B] denotes the concentration of antagonist. K_{D} is the dissociation constant as defined above.

Mean values were compared by Student's t test for unpaired data or by analysis of variance followed by Dunnett's test. A p value< 0.05 was considered significant.

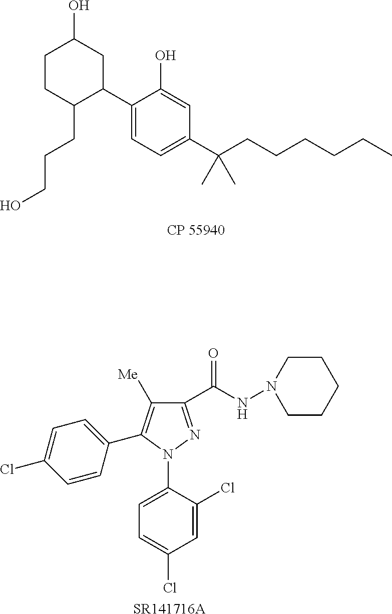

### Binding Assays—Mouse Brain Competition Binding Assay

### Methods

The competition binding assay is a functional radioligand binding assay, which determines the affinity of a given compound for a specific receptor site, in this instance, the cannabinoid CB1 receptor. Mouse brain membranes were the chosen tissue due to their high expression of CB1 receptors.

Whole brains from eight adult male mice were dissected and suspended in centrifugation buffer. The brains were then homogenised with a Polytron Homogeniser prior to centrifugation at 1,600 RCF, 4° C. for 10 minutes. Centrifugation produced a supernatant, which was collected, and then the remaining pellet was re-suspended in centrifugation buffer and centrifuged as before. All supernatants were combined, and centrifuged at 28,000 RCF, 4° C. for a further 20 minutes. The supernatant was then discarded and the pellet re-suspended in Buffer A before incubation for 10 minutes at 37° C. The membrane suspension was then centrifuged at RCF 23,000, 4° C. for 20 minutes. The pellet was then re-suspended in Buffer A and incubated for 40 minutes at room temperature. The suspension was then centrifuged for 15 minutes at RCF 11,000, 4° C., and the pellet was then re-suspended in Buffer B. Finally a Protein Assay was carried out to determine the protein content of the mouse membrane preparation. The membrane samples were then made up into 1 mL aliquots of 1 mg/mL protein concentration and stored at −80° C. The samples were removed and defrosted when they were required.

### Standard Competition Binding Assay Buffer Preparations

The competition binding assay utilizes standard binding buffer, comprising:

### (a) 50 mM Tris HCl;

(b) 50 mM Tris base;

### (c) 0.1% w/v BSA.

### [³H] CP55940 Preparation (0.7 nM Assay Concentration)

2.21 μL of [³H] CP55940 were added to 97.8 μL of binding buffer to make up a 140 nM stock solution. A 7 nM solution was then prepared by the addition of 75 μL of 140 nM stock to 1425 μL of binding buffer.

### Cold CP55940 Preparation (1 μM Assay Concentration)

10 μL of 1 mM stock of CP55940 in DMSO was added to 990 μL of binding buffer.

This was essential to obtain a value for specific binding, which is defined as the difference between the binding that occurs in the presence and absence of 1/μM unlabelled CP55940.

### Vehicle Control (0.1% DMSO in Assay)

10 μL DMSO+ 990/μL of binding buffer.

### Experimental Protocol

Buffer; [³H] CP55940; varying concentrations of SR141716A or test compound; and the mouse brain membranes were pipetted into the appropriate wells of a 96-well plate. The competition assay was initiated by the addition of the membranes. The assay was incubated at 37° C. in a water bath for 1 hour. The assay was terminated by addition of ice-cold Tris/BSA buffer and rapid vacuum filtration using a 24-well Brandel (cell harvester) and glass-fibre filters that had been soaked in Tris/BSA buffer at 4° C. for 24 hours. Each well was washed 6 times with 1.2 mL of Tris/BSA buffer. The filters were then oven dried for 1 hour. The sections of filter papers were then separated and placed in individual vials, to which 5 mL of scintillation fluid was added. The filter papers were soaked in the scintillation fluid for 1 hour before the radioactivity in each vial was quantified by liquid scintillation spectrometry.

### Data Analysis

Data were fitted to a one-site competition curve using GraphPad Prism 4. This gave values for the EC₅₀ expressed as the mean, with variability expressed as S.E.M. or as 95% confidence limits. The dissociation constant (K_{I}-value) of SR141716A and the test compounds was calculated using the equation of Cheng & Prusoff (see, e.g., Yung-Chi Cheng et al., 1973).

### Functional Assays—[5S] GTPγS Binding Assay

The [³⁵S] GTPγS binding assay is simply a means of measuring G protein activation following agonist occupation of a GPCR. The significance of replacing endogenous GTP with radiolabeled [³⁵S] GTPγS is two-fold. The γ-thiophosphate bond is resistant to hydrolysis hence binds irreversibly to the Gα-subunit of the G protein. This results in an accumulation of Gα-[³⁵S] GTPγS. Secondly, as with any radiolabelling, the aim is to allow quantitative analysis of a selected molecular species, in this case the degree of agonist binding can be gauged by measuring the subsequent levels of radioactivity in the desired tissue.

### Standard GTPγS Binding Buffer Preparations

500 mL GTPγS binding buffer was prepared using standard binding buffer, comprising:

### 50 mM Tris HCl;

50 mM Tris base;

### 0.1% w/v BSA;

with the following additional salts:

### 1 mM EDTA (0.188 g);

### 5 mM MgCl₂ (0.238 g);

### 100 mM NaCl (2.922 g);

### 1 mM DTT (0.0775 g).

30 μM GDP was also added. However, since it is unstable in water, it was kept frozen and added on the day of experiment. It was prepared as followed:

1 mM GDP stock (1 mg GDP per 2.256 mL),
1.5 mL of 1 mM stock added to 50 mL Binding buffer.

### [³⁵S] GTPγS Preparation (0.1 nM Assay Concentration)

The [³⁵S] GTPγS was stored in 1 μL aliquots, which were used to make up 100 nM stock source via the addition of 99 μL of binding buffer. From this 100 nM stock, a further dilution was required to attain a 1 nM concentration. This was achieved by adding 15 μL of 100 nM stock to 1485 μL of binding buffer.

### Cold GTPγS Preparation (30 μM Assay Concentration)

The purpose of using cold GTPγS was to allow quantification of non-specific binding. GTPγS undoubtedly binds to sites other than the CB1 receptors upon which their binding was the key interest. By adding cold GTPγS prior to addition of the hot source, the available CB1 receptor sites irreversibly bound the cold GTPγS rendering the receptor unavailable for [³⁵S] GTPγS binding. Hence any binding observed was most likely at an alternative site. This allowed the effect of [³⁵S] GTPγS specifically on CB1 receptors to be reasonably evaluated.

A 1 mM stock was made from 1 mg GTPγS per 1.776 mL of binding buffer (made daily as it cannot be frozen). This 1 mM stock was then further diluted to 300 μM by adding 60 μL of 1 mM stock to 140 μL of binding buffer.

### Vehicle Control (Basal Binding)

A vehicle control was an essential aspect of the experiment. This allows for the constitutive GPCR activity to be measured, hence the specific activity of the subsequent test compound concentrations can be defined. The vehicle is made up of 10 μL of DMSO and 990 μL of binding buffer, resulting in the assay containing 0.1% vehicle.

### Experimental Protocol

Buffer; [³⁵S] GTPγS; varying concentrations of SR141716A or test compound; and the CB1 CHO cells were pipetted into the appropriate wells of a 96-well plate. The GTPγS binding assay was initiated by the addition of the [³⁵S] GTPγS. The assay was incubated at 37° C. in a water bath for 1 hour. The assay was terminated by addition of ice-cold Tris/BSA buffer and rapid vacuum filtration using a 24-well Brandel (cell harvester) and glass-fibre filters that have been soaked in Tris/BSA buffer at 4° C. for 24 hours. Each well was washed 6 times with 1.2 mL of Tris/BSA buffer. The filters were then oven dried for 1 hour. The sections of filter papers were then separated and placed in individual vials, to which 5 mL of scintillation fluid was added. The filter papers were soaked in the scintillation fluid for 1 hour before the radioactivity in each vial was quantified by liquid scintillation spectrometry.

### Data Analysis

Net agonist stimulated [35S] GTPγS-binding values were calculated by subtracting basal binding values (obtained in the absence of agonist) from agonist-stimulated values (obtained in the presence of agonist) (see, e.g., Ross et al., 1999).

The data was fitted to a sigmoidal concentration-response curve using GraphPad Prism 4. Significance was defined by a one-sample t-test (GraphPad Prism 4), which compared mean % stimulation values S.E.M. to the basal binding level, 0.0% stimulation.

### Biological Data

Using the mouse brain competition assay, the [³⁵S] GTPγS binding assay and the mouse vas deferens assay, it has been shown that the compounds described herein are high affinity ligands for the CB1 receptor. Using the [³⁵S] GTPγS binding assay, it has also been shown that the property of inverse agonism has been removed (see the Figures) and that, within the limits of sensitivity of the experiment, these are neutral antagonists. The results are summarised in the following Table.

TABLE 2

| | | K_{D} (nM) | | |
|---|---|---|---|---|
| | | [antagonist potency] | | |
| | Kᵢ (nM) | [against CP55940] | | |
| | [affinity] | Mouse | | Function |
| | Mouse Brain | Brain | Mouse vas | Mouse |
| | [³H]CP55940 | [³⁵S] | deferens | Brain [³⁵S] |
| Compound | Displacement | GTPγS | contractions | GTPγS Alone |
| SR141716A | 1.69 | 0.9 | 3.46 | Inverse agonist |
| ABD395 | 3.78 | 1.4 | 2.35 | Neutral antagonist |
| ABD399 | 12.3 | 3 | 40 | Neutral antagonist |
| ABD402 | 32.3 | 3.8 | 34.8 | Neutral antagonist |
| ABD406 | 24.2 | 2.3 | 32.8 | Neutral antagonist |
| ABD434 | 7.42 | — | — | Neutral antagonist |
| ABD436 | 7.15 | — | — | Neutral antagonist |
| ABD437 | 14.2 | — | — | Neutral antagonist |
| ABD438 | 7.7 | — | — | Neutral antagonist |
| ABD439 | 19.7 | — | — | Neutral antagonist |
| ABD440 | 27.1 | — | — | Neutral antagonist |
| ABD449 | 64.8 | — | — | Neutral antagonist |
| ABD450 | 63.2 | — | — | Neutral antagonist |
| ABD453 | 13.8 | — | — | Neutral antagonist |
| ABD454 | 26.7 | — | — | Neutral antagonist |
| ABD459 | 8.6 | — | 3.92 | Neutral antagonist |

### Biological Study 1

In displacement assays using [³H] CP55940 in mouse brain membranes, SR141716A displaces with a Kᵢ value consistent with a high CB1 receptor affinity (see Table 2).

In the functional assay, (1) [³⁵S] GTPγS and (2) electrically-evoked contraction of the mouse isolated vas deferens, SR141716A causes a right-ward shift in the log concentration response curve for CP55940. The K_{D} values reflect the ability of the compound to act as an antagonist of CB1 receptor agonists (see Table 2).

Electrically-evoked contractions of mouse vas deferens were studied in the presence of the inverse agonist SR141716A or a test compound. The results showed that in this assay the test compounds did not act as inverse agonists and are in fact neutral antagonists.

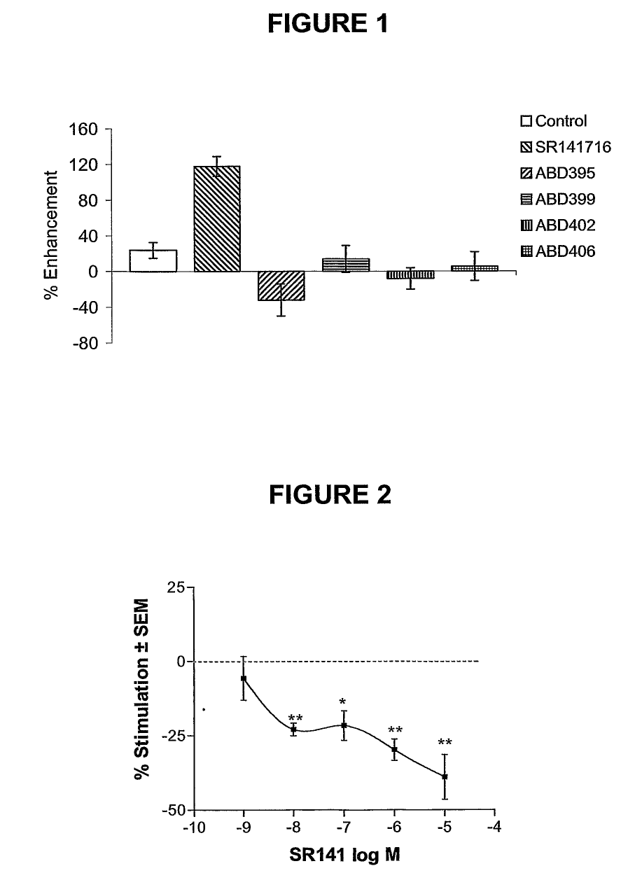

FIG. 1 is a bar graph showing the effect of control (DMSO), 1 μM SR141716A, or 1 μM test compound on electrically-evoked contractions of isolated mouse vas deferens. Each column represents the mean value of the change in the amplitude of the contractions expressed as a percentage of the amplitude measured immediately before the addition of DMSO, 1 μM SR141716A, or 1 μM test compound to the organ bath. The vertical lines indicate S.E.M. (standard error of the mean).

It is well established that SR141716A is a CB1 receptor inverse agonist. This property is reflected in the data shown in FIG. 1. This figure illustrates the significant increase in electrically-evoked contractions of isolated mouse vas deferens when 1 μM SR141716A was tested alone. When tested alone, 1 μM SR141716A significantly increased electrically-evoked contractions of isolated mouse vas deferens. This is indicative of an inverse agonist and is in agreement with the findings of previous mouse vas deferens studies (see, e.g., Price et al., 2005). As shown in FIG. 1, the test compounds, ABD395, ABD399, ABD402, and ABD406 neither inhibited nor significantly enhanced electrically evoked contractions at a concentration of 1 μM. This data suggest that these four SR141716A analogues are neither allosteric agonists nor inverse agonists.

### Biological Study 2—SR141716A

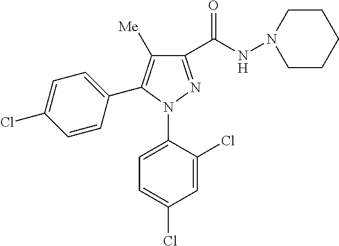

The effects of the known cannabinoid receptor CB1 inverse agonist SR141716A on [³⁵S] GTPγS binding were studied. SR141716A was found to cause a reduction in [³⁵S] GTPγS binding that is indicative of inverse agonism at concentrations of 1 nM and above.

FIG. 2 is a graph showing the effect of different concentrations (1 nM to 10 μM) of SR141716A on [³⁵S] GTPγS binding for SR141716A alone, with 24 hours FBS-starved CB1 CHO cells. The vertical lines indicate S.E.M. (standard error of the mean) (*= P<0.05, **= P<0.01, ***= P<0.001, one-sample t-test) (n=4).

The data show that SR141716A inhibits GDP/GTP turnover, and this reflects the decrease in constitutive activity that is characteristic of an inverse agonist.

### Biological Study 3—ABD395

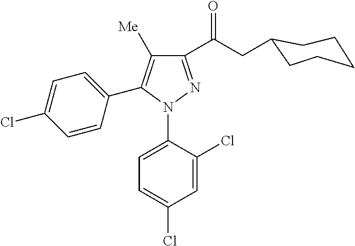

In displacement assays using [³H] CP55940 in mouse brain membranes, ABD395 displaces with a Kᵢ value consistent with a high CB1 receptor affinity (see Table 2).

In the functional assay (1) [³⁵S] GTPγS and (2) electrically-evoked contraction of the mouse isolated vas deferens, ABD395 causes a right-ward shift in the log concentration response curve for CP55940 (see FIG. 4). The K_{D} values reflect the ability of the compound to act as an antagonist of CB1 receptor agonists (see Table 2).

The effect of one test compound (ABD395) on [³⁵S] GTPγS binding was studied. The compound was found to cause only a small increase in [35S] GTPγS binding over the range of concentrations investigated (1 nM to 10 μM), indicative of neutral antagonism.

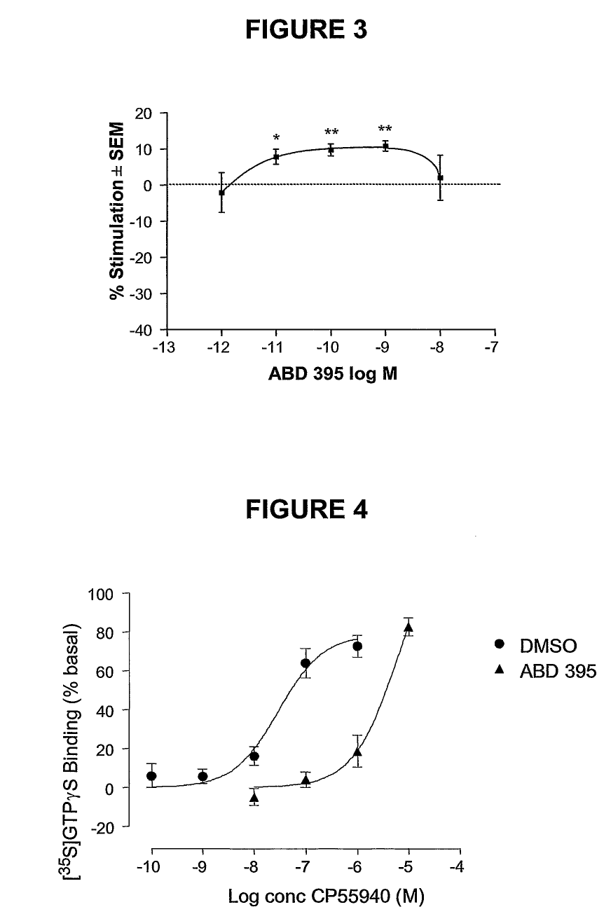

FIG. 3 is a graph showing the effect of different concentrations (1 μM to 10 nM) of ABD395 on [³⁵S] GTPγS binding for ABD395 alone, with 24 hours FBS-starved CB1 CHO cells. The vertical lines indicate S.E.M. (standard error of the mean) (*= P<0.05, **= P<0.01, ***= P<0.001, one-sample t-test) (n= 4).

FIG. 4 is a graph showing the stimulation of [³⁵S] GTPγS in mouse brain membranes by the CB1 receptor agonist, CP55940 (0.1 nM-10,000 nM) in the presence of either DMSO (vehicle) or 300 nM ABD395.

The data show that ABD395 causes only a very weak stimulation of [³⁵S] GTPγS, indicating that it is a neutral antagonist at the CB1 receptor.

### Biological Study 4—ABD399

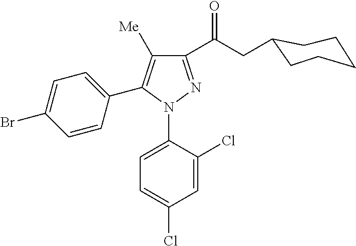

In displacement assays using [³H] CP55940 in mouse brain membranes, ABD399 displaces with a Kᵢ value consistent with a high CB1 receptor affinity (see Table 2).

In the functional assay (1) [³⁵S] GTPγS and (2) electrically-evoked contraction of the mouse isolated vas deferens, ABD399 causes a right-ward shift in the log concentration response curve for CP55940 (see FIG. 6). The K_{D} values reflect the ability of the compound to act as an antagonist of CB1 receptor agonists (Table 2).

The effect of another test compound (ABD399) on [³⁵S] GTPγS binding was studied. The compound was found to cause no reduction in [³⁵S] GTPγS binding over the range of concentrations investigated (1 nM to 10 μM), indicative of neutral antagonism.

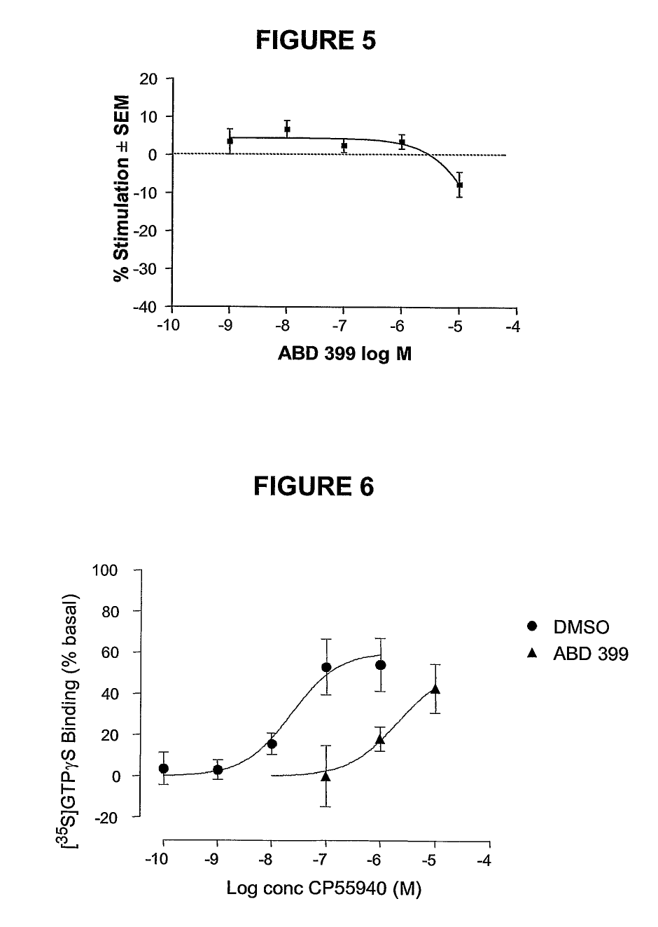

FIG. 5 is a graph showing the effect of different concentrations (1 nM to 10 μM) of ABD399 on [³⁵S] GTPγS binding for ABD399 alone, with 24 hours FBS-starved CB1 CHO cells. The vertical lines indicate S.E.M. (standard error of the mean) (*= P<0.05, **= P<0.01, ***= P<0.001, one-sample t-test) (n= 4).

FIG. 6 is a graph showing the stimulation of [³⁵S] GTPγS in mouse brain membranes by the CB1 receptor agonist, CP55940 (0.1 nM -10,000 nM) in the presence of either DMSO (vehicle) or 300 nM ABD399

The data show that ABD399 produces no change in [³⁵S] GTPγS binding, indicating that it is a neutral antagonist at the CB1 receptor.

### Biological Study 5—ABD402

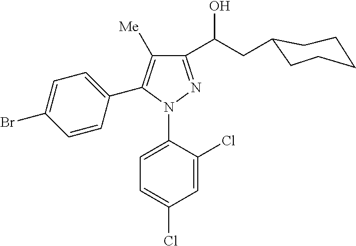

In displacement assays using [³H] CP55940 in mouse brain membranes, ABD402 displaces with a Kᵢ value consistent with a high CB1 receptor affinity (see Table 2).

In the functional assay (1) [³⁵S] GTPγS and (2) electrically-evoked contraction of the mouse isolated vas deferens, ABD402 causes a right-ward shift in the log concentration response curve for CP55940 (see FIG. 8). The K_{D} values reflect the ability of the compound to act as an antagonist of CB1 receptor agonists (see Table 2).

The effect of another test compound (ABD402) on [³⁵S] GTPγS binding was studied. The compound was found to cause no reduction in [³⁵S] GTPγS binding over the range of concentrations investigated (1 nM to 10 μM), indicative of neutral antagonism.

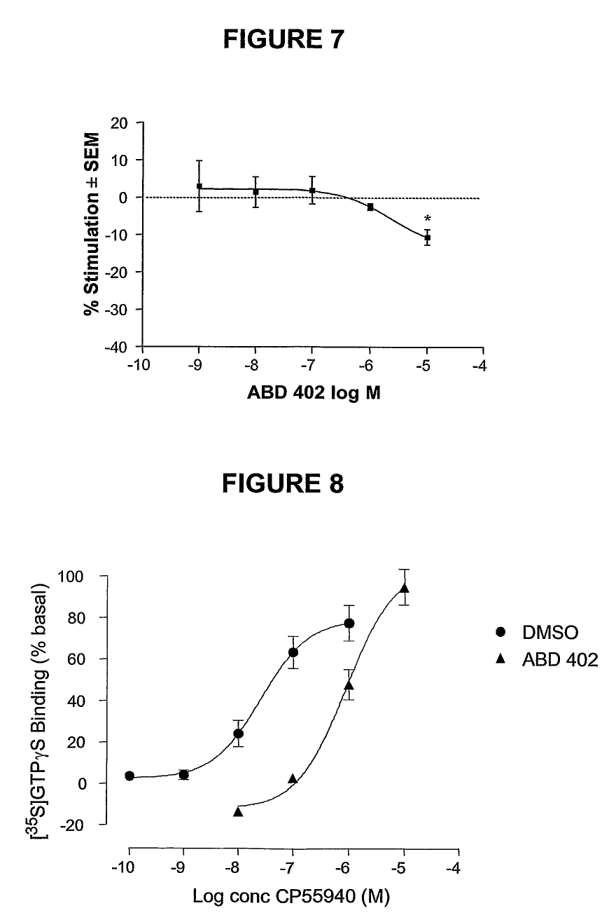

FIG. 7 is a graph showing the effect of different concentrations (1 nM to 10 μM) of ABD402 on [³⁵S] GTPγS binding for ABD402 alone, with 24 hours FBS-starved CB₁A₂ cells. The vertical lines indicate S.E.M. (standard error of the mean) (*= P<0.05, **=P<0.01, ***= P<0.001, one-sample t-test) (n= 4).

FIG. 8 is a graph showing the stimulation of [³⁵S] GTPγS in mouse brain membranes by the CB1 receptor agonist, CP55940 (0.1 nM-10,000 nM) in the presence of either DMSO (vehicle) or 300 nM ABD402.

The data show that ABD402 produces no change in [³⁵S] GTPγS binding, indicating that it is a neutral antagonist at the CB1 receptor.

### Biological Study 6—ABD406

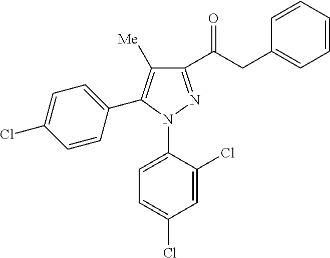

In displacement assays using [³H] CP55940 in mouse brain membranes, ABD406 displaces with a Kᵢ value consistent with a high CB1 receptor affinity (see Table 2).

In the functional assay (1) [³⁵S] GTPγS and (2) electrically-evoked contraction of the mouse isolated vas deferens, ABD406 causes a right-ward shift in the log concentration response curve for CP55940 (see FIG. 10). The K_{D} values reflect the ability of the compound to act as an antagonist of CB1 receptor agonists (see Table 2).

The effect of another test compound (ABD406) on [³⁵S] GTPγS binding was studied. The compound was found to cause no reduction in [³⁵S] GTPγS binding over the range of concentrations investigated (1 nM to 10 μM), indicative of neutral antagonism.

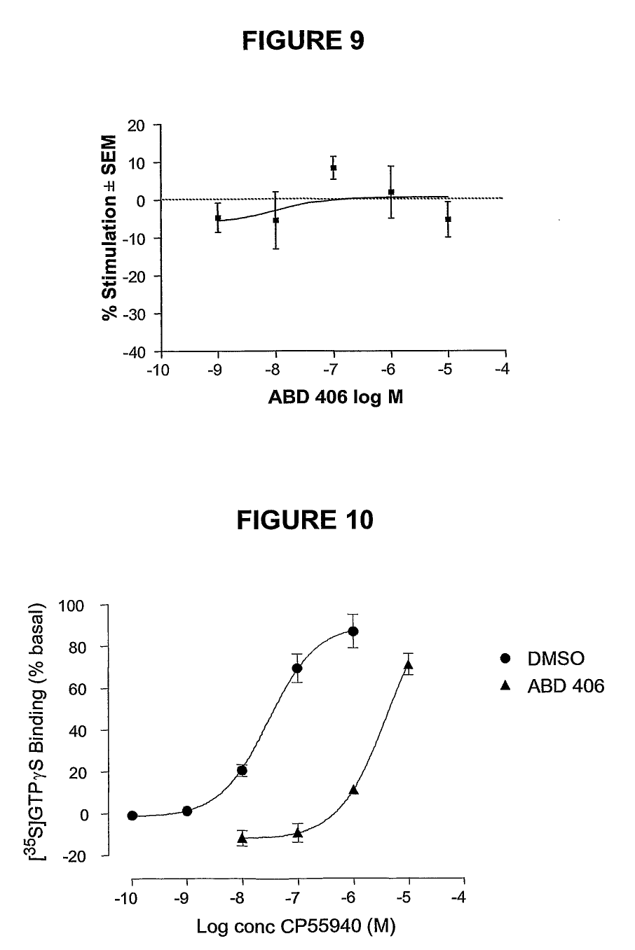

FIG. 9 is a graph showing the effect of different concentrations (1 nM to 10 μM) of ABD406 on [³⁵S] GTPγS binding for ABD406 alone, with 24 hours FBS-starved CB₁A₂ cells. The vertical lines indicate S.E.M. (standard error of the mean) (*= P<0.05, **=P<0.01, ***= P<0.001, one-sample t-test) (n= 4).

FIG. 10 is a graph showing the stimulation of [³⁵S] GTPγS in mouse brain membranes by the CB1 receptor agonist, CP55940 (0.1 nM-10,000 nM) in the presence of either DMSO (vehicle) or 300 nM ABD406.

The data show that ABD406 produces no change in [³⁵S] GTPγS binding, indicating that it is a neutral antagonist at the CB1 receptor.

The foregoing has described the principles, preferred embodiments, and modes of operation of the present invention. However, the invention should not be construed as limited to the particular embodiments discussed. Instead, the above-described embodiments should be regarded as illustrative rather than restrictive, and it should be appreciated that variations may be made in those embodiments by workers skilled in the art without departing from the scope of the present invention as described herein.

### REFERENCES

A number of patents and publications are cited herein in order to more fully describe and disclose the invention and the state of the art to which the invention pertains. Full citations for these references are provided herein. Each of these references is incorporated herein by reference in its entirety into the present disclosure.
Bidaut-Russell, M., Devane, W. A. and Howlett, A. C., 1990, “Cannabinoid receptors and modulation of cyclic AMP accumulation in the rat brain,” J. Neurochem., Vol. 55, pp. 21-26.
Borsche et al., 1939, “Über vielkernige kondensierte Systeme mit heterocyclischen Ringen. IV”, Justus Liebigs Ann. Chem., Vol. 537, p. 219-45.
Bouaboula M, Desnoyer N, Carayon P, Combes T, Casellas P, 1999, “Gi protein modulation induced by a selective inverse agonist for the peripheral cannabinoid receptor CB2: implication for intracellular signalization cross-regulation,” Mol. Pharmacol., Vol. 55, pp. 473-480.
Bouaboula M, Perrachon S, Milligan L, Canat X, Rinaldi-Carmona M, Portier M, Barth F, Calandra B, Pecceu F, Lupker J, Maffrand J P, Le Fur G, Casellas P, 1997, “A selective inverse agonist for central cannabinoid receptor inhibits mitogen-activated protein kinase activation stimulated by insulin or insulin-like growth factor 1. Evidence for a new model of receptor/ligand interactions,” J. Biol. Chem., Vol. 272, pp. 22330-22339.
Bouaboula M, Poinot-Chazel C, Marchand J, Canat X, Bourrie B, Rinaldi-Carmona M, Calandra B, Le Fur G, Casellas P, 1996, “Signaling pathway associated with stimulation of CB2 peripheral cannabinoid receptor. Involvement of both mitogen-activated protein kinase and induction of Krox-24 expression,” Eur. J. Biochem., Vol. 237, pp. 704-711.
Breivogel C S, Griffin G, Di M, V, Martin B R, 2001, “Evidence for a new G protein-coupled cannabinoid receptor in mouse brain,” Mol. Pharmacol. Vol. 60, pp. 155-163.
Callaerts-Vegh et al., 2004, “Effects of acute and chronic administration of beta-adrenoceptor ligands on airway function in a murine model of asthma”, Proc. Natl. Acad. Sci. USA, pp. 4948-4953.
Cheng Y, Prusoff W H, 1973, “Relationship between the inhibition constant (K1) and the concentration of inhibitor which causes 50 percent inhibition (150) of an enzymatic reaction,” Biochem. Pharmacol. Vol. 22, pp. 3099-3108.
Childers, S R, and Deadwyler, S A, 1996, “Role of cyclic AMP in the actions of cannabinoid receptors,” Biochemical Pharmacology, Vol. 52, pp. 819-827.
Colombo et al., 1998, “Appetite suppression and weight loss after the cannabinoid antagonist SR 141716”, Life Sci. Vol. 63, pp. PL113-PL117.
Croci et al., 2003, “Role of cannabinoid CB1 receptors and tumor necrosis factor-alpha in the gut and systemic anti-inflammatory activity of SR 141716 (rimonabant) in rodents.” Brit. J. Pharmacol., Vol. 140, pp. 115-122.
De Ligt et al., 2000, “Inverse agonism at G protein-coupled receptors: (patho)physiological relevance and implications for drug discovery”, Brit. J. Pharmacol. Vol. 130, pp. 1-12.
Derocq J M et al 2000, “Genomic and functional changes induced by the activation of the peripheral cannabinoid receptor CB2 in the promyelocytic cells HL-60. Possible involvement of the CB2 receptor in cell differentiation”, J Biol. Chem. Vol 275, No. 21, pp. 15621-15628.
Fusco et al, 1971, “Reaction of N-pyridinium phenacylids with nitrile imines”, Gazz, Chim. Ital., Vol. 101, p. 703.
Goutopoulos, A, Makriyannis, A., 2002, “From cannabis to cannabinergics: new therapeutic opportunities,” Pharmacology & Therapeutics, Vol. 95, pp. 103-117.
Greig et al., 2004, published international application publication number WO 2004/078261.
Hanus L, Breuer A, Tchilibon S, Shiloah S, Goldenberg D, Horowitz M, Pertwee R G, Ross R A, Mechoulam R, Fride E, 1999, “HU-308: a specific agonist for CB(2), a peripheral cannabinoid receptor,” Proc. Natl. Acad. Sci. U.S.A., Vol. 96, pp. 14228-14233.
Howlett A C, et al., 2002, “International Union of Pharmacology. XXVII. Classification of Cannabinoid Receptors. Pharmacol. Rev., Vol 54 (2), pp. 161-202.
Huffman, J W, 2000, “The Search for Selective Ligands for the CB2 Receptor,” Current Pharmaceutical Design. Vol. 6, pp. 1323-1337.
Huffman, J W, et al., 2001, “A Pyridone Analogue of Traditional Cannabinoids. A New Class of Selective Ligands for the CB2 Receptor,” Bioorganic & Medicinal Chemistry. Vol. 9, pp. 2863-2870.
Hurst et al., 2002, “N-(piperidin-1-yl)-5-(4-chlorophenyl)-1-(2,4-dichlorophenyl)-4-methyl-1H-pyrazole-3-carboxamide (SR141716A) interaction with LYS 3.28(192) is crucial for its inverse agonism at the cannabinoid CB1 receptor”, Mol. Pharmacol., Vol. 62, pp. 1274-1287.
Idris et al., 2005, “Regulation of bone mass, bone loss and osteoclast function by cannabinoid receptors”, Nat. Med., Vol. 11, pp. 774-779.
Lange and Kruse, 2005, “Medicinal chemistry strategies to CB1 cannabinoid receptor antagonists”, Drug Discovery Today, Vol. 10, pp. 693-702.
Lichtman A H, Varvel S A, Martin B R, 2002, “Endocannabinoids in cognition and dependence”, Prostaglandins Leukot. Essent. Fatty Acids. Vol. 66, pp. 269-285.
Martin et al., 2003, U.S. Pat. No. 6,509,367.
Martindale D, 2005, “Can popping a pill curb all your cravings,” New Scientist. 10 Dec. 2005, p. 46.
Pan et al., 1998, “SR 141716A acts as an inverse agonist to increase neuronal voltage-dependent Ca2⁺ currents by reversal of tonic CB1 cannabinoid receptor activity”, Mol. Pharmacol., Vol. 54, pp. 1064-1072.
Parolaro D, Massi P, Rubino T, Monti E, 2002, “Endocannabinoids in the immune system and cancer,” Prostaglandins Leukot. Essent. Fatty Acids, Vol. 66, pp. 319-332.
Pertwee R G, 2005, “Inverse agonism and neutral antagonism at cannabinoid CB₁ receptors”. Life Sci. Vol 76, pp. 1307-1324.
Pertwee R G, Ross RA, 2002, “Cannabinoid receptors and their ligands,” Prostaglandins Leukot. Essent. Fatty Acids. Vol. 66, pp. 101-121.)
Pertwee, R G, 2001, “Cannabinoid Receptor Ligands,” Tocris Reviews, No. 16, April 2001 (publisher Tocris Cookson Inc., USA) (see also http://www.tocris.com, Technical Support and Resources, Life Science Reviews, Cannabinoid Receptor Ligands).
Pertwee, R G, Fernando, S R, 1996, “Evidence for the presence of cannabinoid CB₁ receptors in mouse urinary bladder,” Br. J. Pharmacol., Vol. 118, pp. 2053-2058.
Pi-Sunyer et al., 2006, “Effect of rimonabant, a cannabinoid-1 receptor blocker, on weight and cardiometabolic risk factors in overweight or obese patients: RIO-North America: a randomized controlled trial”, J. Amer. Med. Assoc., Vol. 295, pp. 761-775.
Price M R, Baillie G, Thomas A, Stevenson L A, Easson M, Goodwin R, Walker G, Westwood P, Marrs J, Mclean A, Mcintosh L, Goodwin G, Thompson F, Cowley P, Christopoulos A, Pertwee R G, Ross R A, 2005, “Allosteric Modulation of the Cannabinoid CB₁ Receptor.” Molecular Pharmacology, Vol. 68, pp. 1485-1495.
Rice A S, Farquhar-Smith W P, Nagy I, 2002, “Endocannabinoids and pain: spinal and peripheral analgesia in inflammation and neuropathy,” Prostaglandins Leukot. Essent. Fatty Acids. Vol. 66, pp. 243-256.
Ross R A, Brockie HC, Murphy V L, Templeton F, Makriyannis A, Pertwee R G, 1999, “Agonist-inverse agonist characterisation at CB₁ and CB2 cannabinoid receptors of L759633, L759656 and AM630,” British Journal of Pharmacology, Vol. 126, pp. 665-672.
Ross R A, Brockie H C, Stevenson L A, Murphy V L, Templeton F, Makriyannis A, Pertwee R G, 1999a, “Agonist-inverse agonist characterization at CB1 and CB2 cannabinoid receptors of L759633, L759656, and AM630,” Br. J. Pharmacol. Vol. 126, pp. 665-672.
Ross R A, Gibson T M, Stevenson L A, Saha B, Crocker P, Razdan R K, Pertwee R G, 1999b, “Structural determinants of the partial agonist-inverse agonist properties of 6′-azidohex-2′-yne-delta8-tetrahydrocannabinol at cannabinoid receptors,” Br. J. Pharmacol. Vol. 128, pp. 735-743.
Shawali A S and Abdelhamid A O, 1976, “Reaction of alpha-keto hydrazonyl bromide with carbanions of some active methylene compounds”, J. Heterocycl. Chem. Vol. 13, pp. 989-992.
Shawali A S, 1977, “Synthesis and tautomeric structure of some 2H-pyrazolo[3,4-D]pyridines”, J. Heterocycl. Chem. Vol. 14, p. 375-381.
Soudijn et al., 2005, “Structure-activity relationships of inverse agonists for G-protein-coupled receptors”, Med. Res. Rev., Vol. 25, pp. 398-426.
Vaulquelin et al., 2002, “New Insights into Insurmountable Antagonism,” Fundamental and Clinical Pharmacology,” Vol. 16, pp. 263-272.
Vickers et al., 2003, “Preferential effects of the cannabinoid CB1 receptor antagonist, SR 141716, on food intake and body weight gain of obese (fa/fa) compared to lean Zucker rats”, Psychopharmacol., Vol. 167, pp. 103-111.)
Wadman M., 2006, “Rimonabant adds appetizing choice to slim obesity market”, Nat. Med., Vol. 12, p. 27.
Wiley et al., 2001, “Novel pyrazole cannabinoids: insights into CB(1) receptor recognition and activation”, J. Pharmacol. Exp. Ther. Vol. 296, pp. 1013-1022.

1. A compound selected from compounds of the following formula, and pharmaceutically acceptable salts thereof: 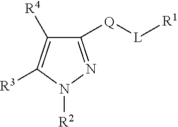
wherein:
Q is independently selected from the following groups: 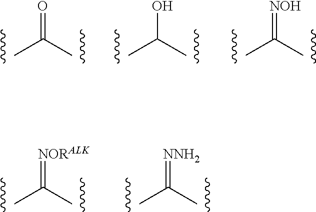
R^{ALK} is independently C₁₋₃alkyl;
L is independently a covalent bond or C₁₋₃alkylene;
R¹ is independently:
C₆₋₁₄carboaryl, and is independently unsubstituted or substituted with one or more ring substituents; or
C₅₋₁₄heteroaryl, and is independently unsubstituted or substituted with one or more ring substituents; or
C₅₋₈cycloalkyl, and is independently unsubstituted or substituted with one or more ring substituents;
R² is independently a group of the following formula, wherein each of R^{2A}, R^{2B}, R^{2C}, R^{2D}, and R^{2E} is independently —H, —Cl, —Br, or —I: 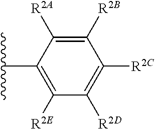
R³ is independently a group of the following formula wherein each of R^{3A}, R^{3B}, R^{3C}, R^{3D}, and R^{3E} is independently —H, —Cl, —Br, or —I: 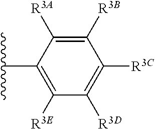
R⁴ is independently Cl₁₋₇alkyl;
wherein each ring substituent, if present, is independently selected from:
(H-1) —C(═O)OH;
(H-2) —C(═O)OR^{a};
(H-3) —C(═O)NH₂, —C(═O)NHR^{a}, —C(═O)NR^{a}R^{a}, —C(═O)NR^{b}R^{c};
(H-4) —C(═O)R^{a};
(H-5) —F, —Cl, —Br, —I;
(H-6) —CN;
(H-7) —NO₂;
(H-8) —OH;
(H-9) —OR^{a};
(H-10) —SH;
(H-11) —SR^{a};
(H-12) —OC(═O)R^{a};
(H-13) —OC(═O)NH₂, —OC(═O)NHR^{a}, —OC(═O)NR^{a}R^{a}, —OC(═O)NR^{b}R^{c};
(H-14) —NH₂, —NHR^{a}, —NR^{a}R^{a}, —NR^{b}R^{c};
(H-15) —NHC(═Ol R^{a}; —NR^{a}C(═O)R^{a};
(H-16; —NHC(═O)NH₂, —NHC(═O)NHR^{a}, —NHC(═O)NR^{a}R^{a}, —NHC(═O)NR^{b}R^{c}, —NR^{a}C(═O)NH₂, —NR^{a}C(═O)NHR^{a}, —NR^{a}C(═O)NR^{a}R^{a}, —NR^{a}C(═O)NR^{b}R^{c};
(H-17) —NHSO₂R^{a}, —NR^{a}SO₂R^{a};
(H-18) —SO₂R^{a};
(H-19) —OSO₂R^{a};
(H-20) —SO₂NH₂, —SO₂NHR^{a}, —SO₂NR^{a}R^{a}, —SO₂NR^{b}R^{a};
(H-21) ═O;
(H-22) —CF₃, and
(H-23) —R^{d};
wherein R^{d} and each R^{a} is independently selected from:
(C-1) C₁₋₇alkyl:
(C-2) C₂₋₇alkenyl;
(C-3) C₂₋₇alkynyl;
(C-4) C₃₋₇cycloalkyl;
(C-5) C₃₋₇cycloalkenyl;
(C-6) C₃₋₁₄heterocyclyl,
(C-7) C₆₋₁₄carboaryl,
(C-8) C₅₋₁₄heteroaryl,
(C-9) C₃₋₇cycloalkyl-C₁₋₃alkylenyl,
(C-10) C₃₋₁₄heterocyclyl-C₁₋₃alkylenyl,
(C-11) C₆₋₁₄carboaryl-C₁₋₃alkylenyl, and
(C-12) C₅₋₁₄heteroaryl-C₁₋₃alkylenyl;
wherein each C₁₋₇alkyl C₂₋₇alkenyl, C₂₋₇alkynyl, C₃₋₇cycloalkyl, C₃₋₇cycloalkenyl, C₃₋₁₄heterocyclyl, C₆₋₁₄carboaryl, and C₅₋₁₄heteroaryl is independently unsubstituted or substituted with one or more substituents selected from (H-1) through (H-22);
and wherein R^{b} and R^{c} taken together with the nitrogen atom to which they are attached form a ring having from 3 to 7 ring atoms.

2-4. (canceled)

5. A compound according to claim 1, wherein Q is independently:

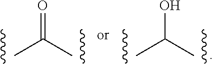

6. A compound according to claim 1, wherein Q is independently:

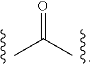

7-9. (canceled)

10. A compound according to claim 1, wherein L is independently a covalent bond.

11. (canceled)

12. A compound according to claim 1, wherein L is independently a covalent bond, —CH₂—, —CH₂CH₂—, or —CH₂CH₂CH₂—.

13-14. (canceled)

15. A compound according to claim 1, wherein L is independently —CH₂—, —CH₂CH₂—, or —CH₂CH₂CH₂—.

16. (canceled)

17. A compound according to claim 1, wherein L is independently —CH₂—.

18-21. (canceled)

22. A compound according to claim 1, wherein R¹ is independently:
C₆₋₁₀carboaryl, and is independently unsubstituted or substituted with one or more ring substituents; or
C₅₋₈cycloalkyl, and is independently unsubstituted or substituted with one or more ring substituents.

23-24. (canceled)

25. A compound according to claim 1, wherein R¹ is independently:
phenyl, and is independently unsubstituted or substituted with one or more ring substituents; or
cyclopentyl, cyclohexyl, cycloheptyl, or cyclooctyl, and is independently unsubstituted or substituted with one or more ring substituents.

26. A compound according to claim 1, wherein R¹ is independently:
phenyl, and is independently unsubstituted or substituted with one or more ring substituents.

27-32. (canceled)

33. A compound according to claim 1, wherein R¹ is independently selected from: 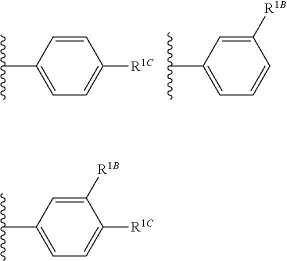
wherein each of R^{1B} and R^{1C}, if present, is independently —H or a ring substituent.

34-39. (canceled)

40. A compound according to claim 33, wherein each of R^{1B} and R^{1C}, if present, is independently —NMe₂, —F, —Cl, —Br, —I, —OH, —OMe, —OEt, -Me, -Et, —CF₃, or —OCF₃.

41. A compound according to claim 1, wherein R¹ is independently:

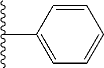

42. A compound according to claim 1, wherein R¹ is independently: 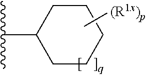
wherein:
p is independently 0, 1, 2, 3, or 4;
q is independently 0, 1, 2, or 3; and
each R^{1X}, if present, is independently a ring substituent.

43-45. (canceled)

46. A compound according to claim 1, wherein R¹ is independently: 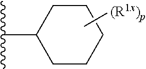
wherein:
p is independently 0, 1, 2, 3, or 4; and
each R^{1X}, if present, is independently a ring substituent.

47-48. (canceled)

49. A compound according to claim 46, wherein each R^{1X}, if present, is independently —NMe₂, —F, —Cl, —Br, —I, —OH, —OMe, —OEt, -Me, -Et, —CF₃, or —OCF₃.

50. A compound according to claim 1, wherein R¹ is independently:

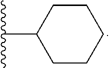

51-53. (canceled)

54. A compound according to claim 1, wherein R₂ is independently: 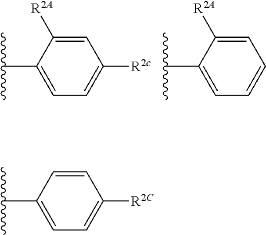
wherein each of R^{2A} and R^{2C}, if present, is independently —H, —Cl, —Br, or —I.

55. (canceled)

56. A compound according to claim 1, wherein R² is independently: 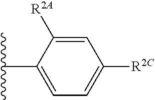
wherein each of R^{2A} and R^{2C} is independently —Cl, —Br, or —I.

57-63. (canceled)

64. A compound according to claim 1, wherein R² is independently:

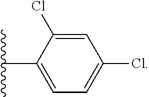

65-67. (canceled)

68. A compound according to claim 1, wherein R³ is independently: 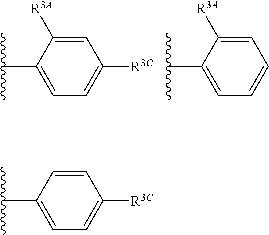
wherein each of R^{3A} and R^{3C}, if present, is independently —Cl, —Br, or —I.

69-73. (canceled)

74. A compound according to claim 1, wherein R³ is independently: 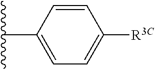
wherein R^{3C} is independently —Cl, —Br, or —I.

75-76. (canceled)

77. A compound according to claim 1, wherein R³ is independently selected from:

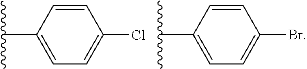

78-79. (canceled)

80. A compound according to claim 1, wherein R⁴ is independently C₁₋₄alkyl.

81. (canceled)

82. A compound according to claim 1, wherein R⁴ is independently -Me.

83-84. (canceled)

85. A compound according to claim 1, wherein each ring substituent, if present, is independently selected from: —C(═O)OH, —C(═O)OMe, —C(═O)OEt, —C(═O)NH₂, —C(═O)NHMe, —C(═O)NHEt, —C(═O)NMe₂, —C(═O)NEt₂, —SO₂Me, —SO₂OH, —NH₂, —NHMe, —NMe₂, —NHEt, —NEt₂, —F, —Cl, —Br, —I, —CN, —NO₂, —OH, —OMe, —OEt, —O(nPr), —O(iPr), —O(cPr), —SH, —SMe, —SEt, -Me, -Et, -nPr -iPr, -cPr, —CF₃, —OCF₃, and ═O.

86. A compound according to claim 1, wherein each ring substituent, if present, is independently selected from: —NMe₂, —F, —Cl, —Br, —I, —CN, —NO₂, —OH, —OMe, —OEt, —O(nPr), —O(iPr), —O(cPr), —SH, —SMe, —SEt, -Me, -Et, -nPr, -iPr, -cPr, —CF₃, and —OCF₃.

87. A compound according to claim 1, wherein each ring substituent, if present, is independently selected from: —NMe₂, —F, —Cl, —Br, —I, —OH, —OMe, —OEt, —O(nPr), —O(iPr), —O(cPr), -Me, -Et, -nPr, -iPr, -cPr, —CF₃, and —OCF₃.

88. A compound according to claim 1, wherein each ring substituent, if present, is independently selected from: —NMe₂, —F, —Cl, —Br, —I, —OH, —OMe, —OEt, -Me, -Et, —CF₃, and —OCF₃.

89. A compound according to claim 1, wherein each ring substituent, if present, is independently selected from: —F, —OMe, -Me, —CF₃, and —OCF₃.

90. A compound according to claim 1, selected from the following compounds, and pharmaceutically acceptable salts:

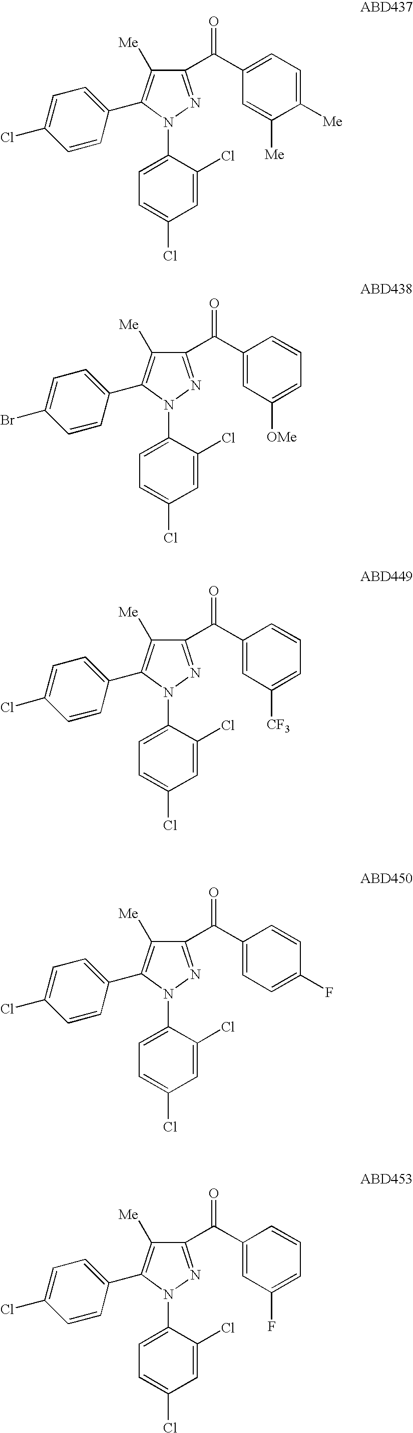

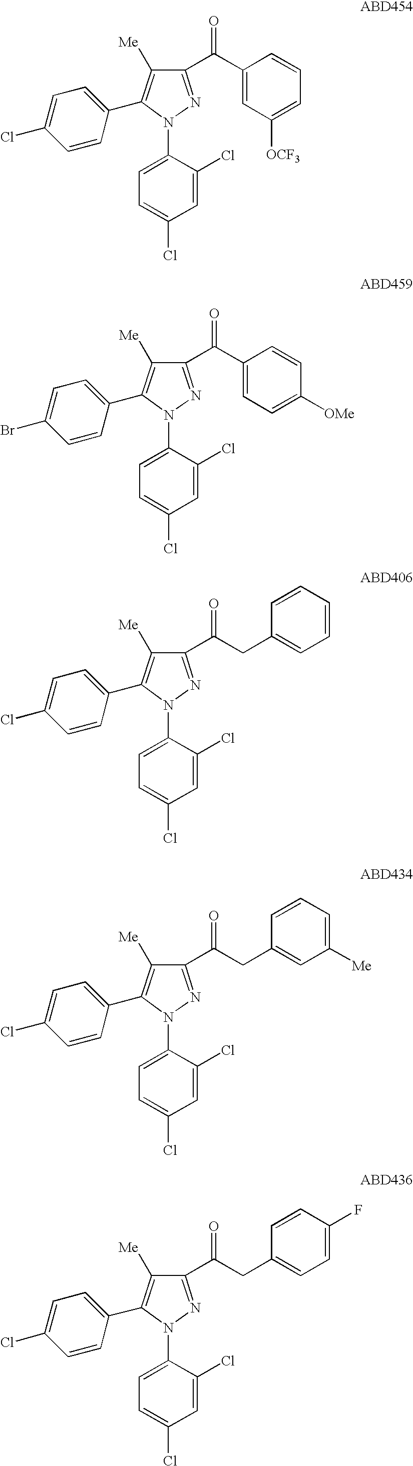

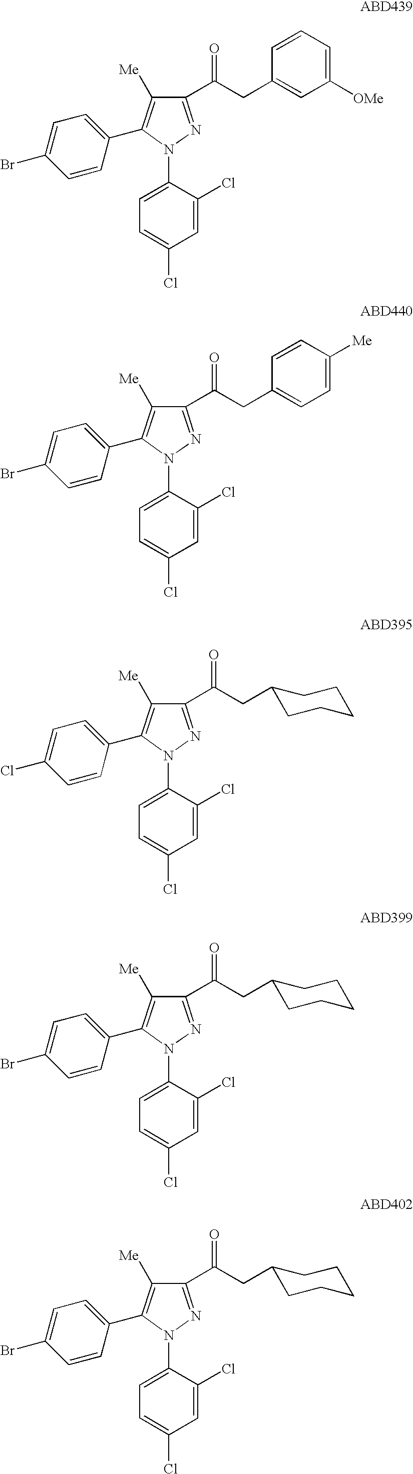

91-92. (canceled)

93. A pharmaceutical composition comprising a compound according to claim 1 and a pharmaceutically acceptable carrier, diluent, or excipient.

94. A method of making a pharmaceutical composition comprising admixing a compound according to claim 1 and a pharmaceutically acceptable carrier, diluent, or excipient.

95-131. (canceled)

132. A method of treatment of a disease or disorder that is ameliorated by treatment with a neutral antagonist of the cannabinoid type 1 (CB1) receptor or a disease or disorder that is associated with activation of the cannabinoid type 1 (CB1) receptor, comprising administering to a patient in need of treatment a therapeutically effective amount of a compound according to claim 1.

133. (canceled)

134. A method of treatment of an eating disorder or obesity, comprising administering to a patient in need of treatment a therapeutically effective amount of a compound according to claim 1.

135. (canceled)

136. A method of treatment of a disease or disorder characterised by an addiction component, addiction, withdrawal, smoking addiction, smoking withdrawal, drug addiction, or drug withdrawal, comprising administering to a patient in need of treatment a therapeutically effective amount of a compound according to claim 1.

137-138. (canceled)

139. A method smoking cessation therapy comprising administering to a patient in need of treatment a therapeutically effective amount of a compound according to claim 1.

140. A method of treatment of a bone disease or disorder, osteoporosis, Paget's disease of bone, or bone related cancer, comprising administering to a patient in need of treatment a therapeutically effective amount of a compound according to claim 1.

141. (canceled)

142. A method of treatment of a disease or disorder with an inflammatory or autoimmune component, rheumatoid arthritis, inflammatory bowel disease, or psoriasis, comprising administering to a patient in need of treatment a therapeutically effective amount of a compound according to claim 1.

143. (canceled)

144. A method of treatment of a psychiatric disease or disorder, anxiety, mania, or schizophrenia, comprising administering to a patient in need of treatment a therapeutically effective amount of a compound according to claim 1.

145. (canceled)

146. A method of treatment of a disease or disorder characterised by impairment of memory and/or loss of cognitive function, memory impairment, loss of cognitive function, Parkinson's disease, Alzheimer's disease, or dementia, comprising administering to a patient in need of treatment a therapeutically effective amount of a compound according to claim 1.

147. (canceled)

148. A method of treatment of a cardiovascular disease or disorder, congestive heart failure, cardiac hypertrophy, or myocardial infarction, comprising administering to a patient in need of treatment a therapeutically effective amount of a compound according to claim 1.

149. (canceled)

150. A compound selected from compounds of the following formula, and pharmaceutically acceptable salts thereof: 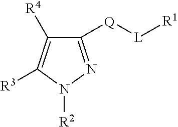
wherein:
Q is independently: 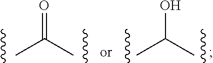
L is independently a covalent bond or —CH₂—;
R¹ is independently:
phenyl, and is independently unsubstituted or substituted with one or more substituents selected from: —C(═O)OH, —C(═O)OMe, —C(═O)OEt, —C(═O)NH₂, —C(═O)NHMe, —C(═O)NHEt, —C(═O)NMe₂, —C(═O)NEt₂, —SO₂Me, —SO₂OH, —NH₂, —NHMe, —NMe₂, —NHEt, —NEt₂, —F, —Cl, —Br, —I, —CN, —NO₂, —OH, —OMe, —OEt, —O(nPr), —O(iPr), —O(cPr), —SH, —SMe, —SEt, -Me, -Et -nPr, -iPr, -cPr, —CF₃, —OCF₃, and ═O; or
cyclopentyl, cyclohexyl, cycloheptyl, or cyclooctyl, and is independently unsubstituted or substituted with one or more substituents selected from: —C(═O)OH, —C(═O)OMe, —C(═O)OEt, —C(═O)NH₂, —C(═O)NHMe, —NMe₂, —NHEt, —NEt₂, —C(═O)NMe₂, —C(═O)NEt₂, —SO₂Me, —SO₂OH, —NH₂, —NHMe, —NMe₂, —NHEt, —NEt₂, —F, —Cl, —Br, —I, —CN, —NO₂, —OH, —OMe, —OEt, —O(nPr), —O(iPr), —O(cPr), —SH, —SMe, —SEt -Me, -Et, -nPr, -iPr, -cPr, —CF₃, —OCF₃, and ═O;
R² is independently: 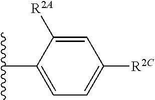
wherein each of R^{2A} and R^{2C} is independently —Cl or —Br;
wherein R³ is independently: 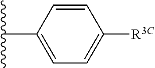
wherein R^{3C} is independently —Cl or —Br; and
R⁴ is independently C₁₋₄alkyl.

151. A compound selected from compounds of the following formula, and pharmaceutically acceptable salts thereof: 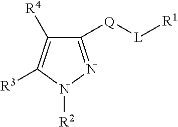
wherein:
Q is independently: 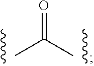
L is independently a covalent bond;
R¹ is independently:
phenyl, and is independently unsubstituted or substituted with one or more substituents selected from: —NMe₂, —F, —Cl, —Br, —I, —OH, —OMe, —OEt, —O(nPr), —O(iPr), —O(cPr), -Me, -Et, -nPr, -iPr, -cPr, —CF₃, and —OCF₂;
R² is independently: 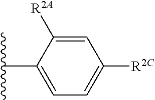
wherein each of R^{2A} and R^{2C} is independently —Cl;
wherein R³ is independently: 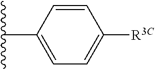
wherein R^{3C} is independently —Cl, or —Br; and
R⁴ is independently -Me.
